(19) United States
(12) Patent Application Publication
(10) Pub. No.: US 2026/0341485 A1
(43) Pub. Date: Oct. 29, 2026

(54) REINFORCEMENT LEARNING DRIVEN OPTIMIZATION SYSTEM

(71) Applicant: Bloom Value Corporation, Norwalk, CT (US)

(72) Inventors: Sampoorna HEGDE, Bengaluru (IN); Yashpal Dhawle, Bengaluru (IN); Priyanka Raj, Bengaluru (IN); Nadeem Ahmed Ayaz, Bengaluru (IN); Laxmanrao Meesala, Bengaluru (IN); Nandini D, Bengaluru (IN); Shashank Conjeevaram, Bengaluru (IN)

(21) Appl. No.: 19/653,949

(22) Filed: Apr. 21, 2026

(30) Foreign Application Priority Data
Apr. 23, 2025 (IN) 202541038810

(51) Int. Cl. G16H 40/20; G06F 16/25; G06F 40/295; G06N 20/00

(52) U.S. Cl. CPC G16H 40/20; G06F 16/254; G06F 40/295; G06N 20/00

(57) ABSTRACT

A system and method for data processing is described. In one aspect, the system implementing the method steps executes operations to extract historical transactional records from healthcare data systems via an ETL pipeline and maps coding nomenclatures into a standardized format. A PCA projects the records into an orthogonal lower-dimensional feature space. Machine learning engines construct a procedure-revenue profile matrix that identifies code pairings with mean transaction outcome values exceeding zero and maps usage metrics. Candidate code pairs are validated against structural and regulatory rules, and violating pairs are filtered. A hierarchical calculation engine determines the predicted outcome value based on temporal attributes and priority hierarchy. A reinforcement learning engine executing a multi-armed bandit algorithm adjusts its weight scores based on reward signals from downstream outcome data. The system transmits structured data output to an external data system via an API.

### CROSS REFERENCE

This application claims the benefit of an Indian provisional patent application filed on Apr. 23, 2025, in the Indian Patent Office and assigned an application number 202541038810, titled “SYSTEM AND METHOD FOR LARGE-SCALE AUTOMATED PAYMENT AND CONTRACT COMPLIANCE AUDITS,” which is hereby incorporated by reference in its entirety.

### FIELD

Various embodiments of the disclosure relate to computational systems for combinatorial optimization in large-scale transactional data environments. Specifically, the present disclosure relates to a system and method that implements mechanisms to manage the computationally intractable search space arising from multi-dimensional code pairing across diverse payer contracts, service locations, and time-based rate windows in healthcare systems.

### BACKGROUND

Medical coding and reimbursement are crucial functions in the healthcare industry, directly impacting the financial stability of healthcare providers and the efficiency of the healthcare system. As healthcare delivery becomes more complex, accurately assigning procedure codes, revenue codes, and billing parameters to individual claims has grown more challenging. Traditional coding and billing methods have depended heavily on manual processes, individual coder expertise, and static rule-based systems. These approaches are inherently limited in their ability to manage the multi-dimensional variability caused by different payer contracts, location-specific rules, patient classifications, timing of rate changes, and new regulatory requirements.

Conventional methods for pairing procedure and revenue codes in healthcare claims require evaluating each valid combination across a multi-dimensional space defined by procedure codes, revenue codes, payer IDs, service locations, patient types, and varying contract rate periods. For a typical hospital network processing claims with about 5,000 procedure codes, 800 revenue codes, 40 payers, and 15 locations, this candidate combination space exceeds 2.4 billion entries per billing cycle. Each combination must also be checked against payer-specific rate schedules that change across overlapping effective dates, further expanding the search space in time. This exponential growth makes exhaustive manual review or static rule-based evaluation computationally impractical within relevant processing timeframes. The issue is not merely about improving human workflow efficiency but about addressing a computational scaling challenge inherent to the data's high dimensionality, similar to well-known combinatorial problems in computer science where brute-force enumeration becomes impossible as input size grows. Existing external data systems offer healthcare claim editing to capture syntax errors and basic code checks, but do not perform payer-specific, contract-rate-aware optimization across the full space of procedure and revenue codes. The lack of native reconciliation at the claim line level between contract rates and actual payments leads to systematic revenue discrepancies that go unnoticed for months after service completion.

Existing external data platforms (e.g., healthcare-related data platforms) have been found to lack native capabilities for several important revenue cycle functions. Neither major external data platform natively compares actual reimbursement received against contracted rates at the claim line level. Contract variances are typically discovered months after the date of service, resulting in an estimated 0.5 to 2 percent of net patient revenue being lost annually to undetected underpayments. Manual review of explanation-of-benefits documents for underpayment detection is impractical at institutional billing volumes, with most healthcare organizations recovering fewer than 30 percent of recoverable underpayments under current workflows.

Accordingly, there is a need for a system and method that addresses the computational intractability of multi-dimensional procedure-revenue code optimization, performs automated reconciliation of contracted rates against actual payments at the claim line level, and provides transparency and auditability of its recommendations across data extracted from multiple disparate billing systems.

Further limitations and disadvantages of conventional and traditional approaches will become apparent to one of skill in the art through comparison of such systems with some aspects of the present disclosure as set forth in the remainder of the present application with reference to the drawings.

### SUMMARY

A method, a system, and a non-transitory computer-readable medium for a reinforcement-learning-driven profile matrix optimization system are described. In an embodiment, the system implements the method steps described herein. The reinforcement-learning-driven profile matrix optimization system may be interchangeably referred to as a system, an integrated system, a data processing system, etc. These and other systems, methods, features, and advantages of the present disclosure will be or become apparent to one having ordinary skill in the art upon examination of the following drawings and detailed description. It is intended that all such additional systems, methods, features, and advantages be included in this description, be in the scope of the present disclosure, and be protected by the accompanying claims.

### BRIEF DESCRIPTION OF THE DRAWINGS

FIG. 1 illustrates a block diagram of an environment, including a reinforcement-learning driven profile matrix optimization system, according to an exemplary embodiment.

FIG. 2A and FIG. 2B illustrates a code combination optimization engine, according to an exemplary embodiment.

FIG. 3 illustrates a block diagram showing a rate card and plans data structure, according to an exemplary embodiment.

FIG. 4 illustrates a block diagram showing a medical code combination optimization system, according to an exemplary embodiment.

FIG. 5 illustrates a flow diagram showing a data flow process for medical claim processing, according to an exemplary embodiment.

FIG. 6 illustrates a block diagram showing a consolidated system for rates, claims, and query preparation execution, according to an exemplary embodiment.

FIG. 7 illustrates a database schema for the reinforcement-learning-driven profile matrix optimization system, according to an exemplary embodiment.

FIG. 8 illustrates a block diagram showing a query preparation and execution system for medical claims processing, according to an exemplary embodiment.

FIG. 9 illustrates a block diagram showing a worked example of the combinatorial claims optimization mechanism, according to an exemplary embodiment.

FIG. 10 illustrates a user interface showing a ranking mechanism for prioritizing and processing medical claim code combinations, according to an exemplary embodiment.

FIG. 11 illustrates a flow diagram showing a process for reinforcement learning feedback loop, according to an exemplary embodiment.

FIG. 12 illustrates a mechanism for updating coding standards, according to an exemplary embodiment.

FIG. 13 illustrates an end-to-end claim processing pipeline, according to an exemplary embodiment.

FIG. 14 illustrates a block diagram showing a per-hospital data processing pipeline, according to an exemplary embodiment.

FIG. 15A and FIG. 15B illustrates a side-by-side comparison between a manual process and a system-assisted process for medical claim coding and submission, according to an exemplary embodiment.

FIG. 16 illustrates a deployment architecture of the reinforcement-learning-driven profile matrix optimization system, according to an exemplary embodiment.

FIG. 17A and FIG. 17B illustrates a flow diagram showing a process implemented by the reinforcement-learning-driven profile matrix optimization system, according to an exemplary embodiment.

FIG. 18 illustrates an exemplary hardware configuration of a computer used to implement the system and method described herein, according to exemplary embodiments.

### DETAILED DESCRIPTION

In the following description, for purposes of explanation, numerous specific details are set forth to provide a thorough understanding of the present disclosure. It will be apparent, however, to one skilled in the art that the present disclosure can be practiced without these specific details. In other instances, systems, apparatuses, and methods are shown in block diagram form only to avoid obscuring the present disclosure.

Reference in this specification to “one embodiment” or “an embodiment” or “example embodiment” means that a particular feature, structure, or characteristic described in connection with the embodiment is included in at least one embodiment of the present disclosure. The appearance of the phrase “in one embodiment” in various places in the specification does not necessarily all refer to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described that may be exhibited by some embodiments and not by others. Similarly, various requirements are described, which may apply to some embodiments but not to others.

The data processing system that implements the method steps addresses the combinatorial intractability problem identified above by implementing a technical architecture that reduces the effective search space, dynamically updates or modifies the combination weights based on empirical outcome data, and resolves rate-card conflicts through a deterministic hierarchical priority mechanism. The data processing system provides technical improvements, including reducing the dimensionality of the input feature space through principal component analysis applied to per-payer and per-location partitions, retaining only those principal components that collectively explain at least 95% of the observed variance, thereby eliminating redundant and non-discriminative features before any profile generation occurs. The data processing system may improve the overall functioning of the computing system.

In an embodiment, constructing a procedure-revenue profile (PRP) matrix may include storing code pairings that exceed an auto-calculated minimum sample threshold, and/or those indexed by procedure code, revenue code, and payer identifier. Such storage logic or mechanism enables the compression of the billions-scale candidate space into a tractable, sparse matrix structure. Further, applying a contextual bandit reinforcement learning mechanism that continuously updates the weight assigned to each matrix cell based on a composite reward signal derived from actual downstream transaction outcomes, approval status, and constraint-violation penalties, using an Upper Confidence Bound (UCB) selection mechanism that balances exploitation of high-reward combinations against exploration of under-sampled combinations. Further, the data processing system resolves temporal rate-card conflicts by selecting the applicable rate record based on service-date temporal matching, followed by a specificity hierarchy of plan-specific over location-specific over general rate, and avoiding exhaustive pairwise comparison of all overlapping rate windows.

The terms “comprise,” “comprising,” “includes,” or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus preceded by “comprises . . . a” do not, without more constraints, preclude the existence of other elements or additional elements in the system or method.

The terms machine learning model or machine learning engine may refer to a computational, statistical, or mathematical model or engine trained using machine learning techniques. The machine learning model or engine may be trained on a dataset using an algorithm to learn, or on a model derived from the dataset. The term artificial intelligence may refer to a machine learning model built using simple or complex neural networks, deep learning techniques, and computer vision algorithms. An artificial intelligence model is trained on data, learns from it, and applies that learning to achieve specific pre-defined objectives.

The term “module” refers to an independent functional unit of software instructions stored in memory and executable by one or more processors to perform a specific set of operations on input data and produce output data. The term “engine” refers to a computational component that implements algorithms, mechanisms, processes, mathematical models, or decision logic to process, transform, evaluate, or optimize data, and that may invoke other modules or engines during execution. The term “subsystem” refers to an organized grouping of modules or engines that collectively perform a higher-level processing task within a system. The term “component” generally refers to any identifiable structural or functional element of the system that may operate independently or in cooperation with other elements, such as a module, an engine, a subsystem, a data store, an interface, or a communication pathway.

The term reinforcement-learning-driven profile matrix optimization system may refer to either an integrated computational system or a data processing system that implements the elements described herein. The data processing system executes operations to reduce a combinatorial search space of candidate code pairs through dimensionality reduction, matrix-based profiling, and reinforcement-learning-driven weight adjustment.

The term ‘optimization’ may refer to a computational process of traversing a combinatorial search space of candidate code pairs, the search space defined by the Cartesian product of N procedure codes, M revenue codes, P payer identifiers, and L location identifiers, and selecting from that search space one or more candidate code pairs satisfying structural and regulatory constraints while maximizing an adjusted weight score computed by a reinforcement learning engine. The optimization may be performed on a procedure-revenue profile matrix using operations such as, but not limited to, dimensionality reduction, constraint filtering, and iterative weight modifications or adjustments.

As used herein, a ‘hospital billing system’ is an example of a ‘healthcare data system’ or a ‘transactional data system.’ A ‘reimbursement value’ is an example of an ‘outcome value’ or a ‘transaction outcome value.’ An ‘expected reimbursement output’ is an example of an ‘expected outcome value’ or a ‘predicted outcome value.’ An ‘Electronic Health Record (EHR) system’ is an example of an ‘external data system.’

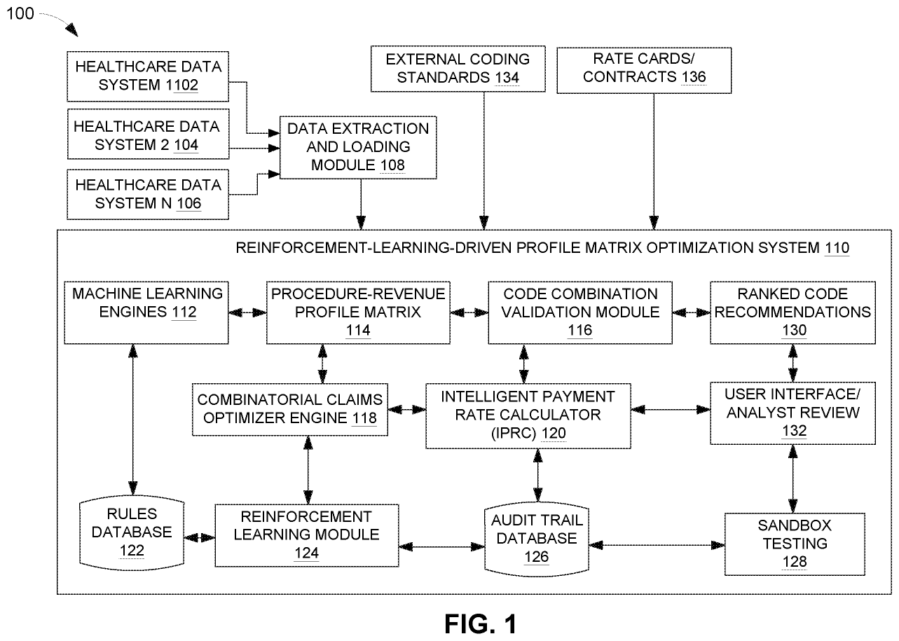

FIG. 1 illustrates a block diagram of an environment 100 including a reinforcement-learning-driven profile matrix optimization system, according to an exemplary embodiment. FIG. 1 illustrates a reinforcement-learning-driven profile matrix optimization system 110. In an embodiment, the reinforcement-learning-driven profile matrix optimization system 110 may include a hospital billing system 102, a hospital billing system 2 104, a hospital billing system N 106, a data extraction and loading module 108, a reinforcement-learning-driven profile matrix optimization system 110, machine learning engines 112, a PRP matrix 114, a code combination validation module 116, a combinatorial claims optimizer engine 118, an intelligent payment rate calculator (iPRC) 120, a rules database 122, a reinforcement learning module 124, an audit trail database 126, a sandbox testing module 128, ranked code recommendations 130, a user interface/analyst review module 132, external coding standards 134, and rate cards/contracts 136.

In an embodiment, the healthcare data systems 1 102, 2 104, and N 106 may represent multiple hospital billing systems from which the reinforcement-learning-driven profile matrix optimization system 110 may receive historical transactional records. Each hospital billing system (e.g., 102, 104, and 106) may generate data using different coding nomenclatures, data formats, and schema structures. The healthcare data systems (e.g., 102, 104, and 106) may transmit data using healthcare data interchange standards and may include hospital information systems, such as those provided by external data vendors (e.g., healthcare-related data).

The data extraction and loading module 108 may interface with each of the healthcare data systems (e.g., 102, 104, and 106) using, for example, an extract, transform, load (ETL) pipeline. The data extraction and loading module 108 may receive historical transactional records from the healthcare data systems (e.g., 102, 104, and 106) and map the hospital coding nomenclature, data formats, and schema structures into a standardized format. For example, the standardized format produced may include a procedure code, a revenue code, a payer identifier, and a transaction outcome value (e.g., reimbursement value or reimbursement amount) for each transactional record. The data extraction and loading module 108 may store the standardized transactional records in columnar storage partitions of distributed databases. The technical advantage of the ETL-based extraction with columnar partitioning is that the data processing system facilitates processing large volumes of medical claims data from multiple hospital systems without manual schema reconciliation.

In an embodiment, the data extraction and loading module 108 may be implemented using the ETL pipelines that execute operations to extract data from the healthcare data systems (e.g., 102, 104, and 106). The data extraction and loading module 108 may implement data mapping techniques to associate the coding nomenclature of each hospital billing system (e.g., 102, 104, and 106) with standardized medical coding systems such as the International Classification of Diseases (ICD) and Current Procedural Terminology (CPT). The data extraction and loading module 108 may execute operations that enable code optimization before claim submission to payers, or post-submission audits of previously submitted claims.

The reinforcement-learning-driven profile matrix optimization system 110 may include the machine learning engines 112, the PRP matrix 114, the code combination validation module 116, the combinatorial claims optimizer engine 118, the iPRC 120, the rules database 122, the reinforcement learning module 124, the audit trail database 126, the sandbox testing module 128, and the ranked code recommendations 130. The reinforcement-learning-driven profile matrix optimization system 110 may execute operations related to a combinatorial search space exceeding N*M*P*L candidate code pairs, where N may be the number of procedure codes, M may be the number of revenue codes, P may be the number of payers, and L may be the number of locations, resulting in a search space that may be computationally intractable for manual or rule-based approaches. For example, in a deployment scenario for a mid-size hospital network processing medical claims across 5,000 procedure codes, 800 revenue codes, 40 payers, and 15 locations, the resulting search space may exceed 2.4 billion candidate combinations per billing cycle. In various embodiments, the multiple historical transactional records may be associated with at least 500, at least 1,000, at least 2,000, at least 3,000, at least 4,000, or at least 5,000 procedure codes. In various embodiments, the multiple historical transactional records may be associated with at least 100, at least 200, at least 400, at least 600, or at least 800 revenue codes. In various embodiments, the multiple historical transactional records may be associated with at least 10, at least 20, at least 30, or at least 40 payers. In various embodiments, the multiple historical transactional records may be associated with at least 5, at least 10, or at least 15 service locations. In various embodiments, the resulting combinatorial search space may exceed at least 100 million, at least 500 million, at least 1 billion, or at least 2 billion candidate code pair combinations.

The machine learning engines 112 may execute operations to process historical transactional records received from the data extraction and loading module 108 in the standardized format and to construct the PRP matrix 114. The machine learning engines 112 may execute operations related to association rule mining to identify statistically significant correlations between procedure codes and revenue codes from the historical claims data. The machine learning engines 112 may include a principal component analysis (PCA) module or component (not shown) that may execute operations to project historical transactional records into an orthogonal, lower-dimensional feature space and generate a subset of principal components that satisfy a predetermined variance threshold. For example, a predetermined threshold of approximately 95% may be applied by the machine learning engines 112. The projection into the orthogonal, lower-dimensional feature space performed by the PCA module may be applied to each partition of the historical transactional records defined by the payer identifier and the location identifier, thereby accounting for payer-specific billing patterns. The machine learning engines 112 may execute integer programming to optimize combinatorially candidate code pairs derived from the PRP matrix 114. The technical advantage is that the PCA module eliminates redundant and collinear features before the machine learning engines construct the procedure-revenue profile matrix, reducing computational overhead and preventing redundant data from degrading the quality of the profile matrix.

The machine learning engines 112 may implement multi-stage architectures (e.g., a first machine learning engine, a second machine learning engine, a third machine learning engine, etc.). For example, the machine learning engines 112 may implement a two-stage architecture in which a first machine learning engine may perform code combination learning from historical data using linear regression, and a second machine learning engine may perform code combination recommendation for new claims using integer programming. The machine learning engines 112 may identify procedure codes, each paired with a revenue code. The identified procedure codes may have a mean transaction outcome value (e.g., reimbursement value) exceeding zero across records associated with non-zero transaction outcome values, and may map a usage metric and a weight score to a corresponding cell of the PRP matrix 114 for each identified pairing. The machine learning engines 112 may execute operations to filter procedure codes that have only one associated revenue code. The machine learning engines 112 may execute operations to implement threshold pruning by auto-calculating a minimum sample count and eliminating each identified pairing of procedure codes and revenue codes that may be associated with fewer than the minimum number of records with non-zero transaction outcome values. The minimum sample count may be auto-calculated by the machine learning engines 112, and combinations with fewer than three sample data instances may be eliminated.

The procedure-revenue profile (PRP) matrix 114 may be a data structure stored in distributed databases and constructed by the machine learning engines 112. For each identified pairing of a procedure code and a revenue code, the PRP matrix 114 may store a usage metric and a weight score in the corresponding cell. Each cell may be indexed for retrieval by a combination of the procedure code, the revenue code, and the payer identifier. The PRP matrix 114 may be stored in the columnar storage partitions as a partitioned data structure including fields associated with the usage metric, the weight score, an exploration-exploitation score computed by the reinforcement learning module 124 for each validated candidate code pair, an effective date, and a status indicator. The PRP matrix 114 may be partitioned by the payer identifier such that a given procedure code may have a different weight score for a first payer identifier than for a second payer identifier. The PRP matrix 114 may be structured including data fields such as a profile identifier as a primary key, a procedure code, a revenue code, a payer identifier, a location identifier, a patient class, a usage percentage, an approval rate, an average transaction outcome value, a weight score, an effective start date, an effective end date, a last updated timestamp, and a model version, with a unique constraint on the combination of procedure code, revenue code, payer identifier, and location identifier.

The code combination validation module 116 may execute operations to validate candidate code pairs retrieved from the PRP matrix 114 against predefined coding rules stored in the rules database 122. The code combination validation module 116 may apply structural rules and regulatory rules to each candidate code pair. The structural rules applied by the code combination validation module 116 may include domain-specific rules that define valid relationships between procedures and treatment locations, and inter-procedure dependency constraints that verify revenue codes selected for a first procedure code do not conflict with revenue codes selected for a second procedure code on the same transactional record. The code combination validation module 116 may include a location-constraint checker configured to apply the structural rules to verify that a first procedure code may be permissible for a treatment location identified in a target transactional record. The dependency-constraint checker is configured to apply the structural rules to verify that the revenue codes selected for the first procedure code on the target transactional record do not conflict with the revenue codes selected for a second procedure code on the same target transactional record.

The regulatory rules applied by the code combination validation module 116 may include rules sourced from the Centers for Medicare and Medicaid Services (CMS), the American Medical Association (AMA), state-specific Medicaid requirements, and individual payer-specific requirements. The code combination validation module 116 may filter candidate code pairs to eliminate each candidate code pair that may violate a structural rule or a regulatory rule. Based on a confidence score, the code combination validation module 116 may classify each validated candidate code pair into one of three categories, for example, auto-approved, auto-rejected, or flagged-for-manual-review. The confidence score used by the code combination validation module 116 to classify each validated candidate code pair may be derived from the weight score and usage metric stored in the PRP matrix 114 for that candidate code pair. Candidate code pairs flagged for manual review may be routed to the user interface/analyst review module 132 for human assessment by a revenue cycle analyst.

The combinatorial claims optimizer engine 118 may execute operations to process a new medical claim by identifying applicable procedure codes and generating multiple valid procedure-revenue code combinations based on the PRP matrix 114. The combinatorial claims optimizer engine 118 may select an optimal code combination that maximizes reimbursement while maintaining compliance with predefined coding rules stored in the rules database 122. The combinatorial claims optimizer engine 118 may execute operations by cross-joining each claim with the profiled combinations from the PRP matrix 114 to generate a matrix of candidate code variants. For a claim with ‘N’ procedure codes, each having ‘M’ possible revenue codes, the combinatorial claims optimizer engine 118 may manage a theoretical search space of M^{N} combinations. For each candidate code combination, the combinatorial claims optimizer engine 118 may receive predicted outcome values (e.g., expected reimbursement output) from the iPRC 120 and may rank the candidate code combinations by expected transaction outcome value, subject to validation constraints received from the code combination validation module 116. The output of the combinatorial claims optimizer engine 118 may be the ranked code recommendations 130, which may be transmitted to the user interface/analyst review module 132.

The iPRC 120 may execute operations to calculate expected reimbursement rates for each validated candidate code pair generated by the combinatorial claims optimizer engine 118. The iPRC 120 may determine a predicted outcome value (e.g., expected reimbursement output) for each validated candidate code pair using a hierarchical calculation engine. The hierarchical calculation engine may execute operations to select a rate record from a rate data store based on a temporal attribute of a target transactional record and a priority hierarchy, and that applies a rate formula selected based on a combination of the payer identifier, a plan identifier, and a location identifier associated with the target transactional record. The priority hierarchy of the iPRC 120 may prioritize a plan-specific rate record over a location-specific rate record, and then over a general rate record. The iPRC 120 may implement multiple payment mechanisms, and the rate formula may be selected from a per-case formula (e.g., calculated as base rate multiplied by diagnosis-related group (DRG) weight multiplied by location adjustment), a per-diem formula (e.g., calculated as daily rate multiplied by length of stay, capped at a contractual maximum), a diagnosis-group-based percentage-of-charges formula (e.g., calculated as total charges multiplied by a contracted percentage), a per-unit formula (e.g., calculated as the sum of unit rate multiplied by units for each line item), and a fee-schedule formula (e.g., determined by a fee schedule rate looked up by procedure code, location, and effective date). The iPRC 120 may resolve conflicts when multiple rate cards apply to a single claim by using temporal priority. The service date is matched to the effective date range, followed by the specificity hierarchy described above. The iPRC 120 may include a rate exception engine that executes operations to evaluate multiple exception rules across multiple plans, service locations, and contracts stored in the rate data store. The rate exception engine of the iPRC 120 may process, for example, 772,546 or more health plan rules across 71 plans, 12 locations, and 39 contracts to identify applicable rate exceptions for a given validated candidate code pair. The iPRC 120 may receive rate card data and payer contract data from the rate cards/contracts 136 data source. In various embodiments, the rate exception engine may process at least 100,000, at least 250,000, at least 500,000, or at least 750,000 health plan rules. In various embodiments, the rate exception engine may process rules across at least 20, at least 40, at least 60, or at least 70 plans. In various embodiments, the rate exception engine may process rules across at least 10, at least 20, at least 30, or at least 40 contracts.

The rules database 122 may store predefined coding rules against which the code combination validation module 116 may validate candidate code pairs. The rules database 122 may store structural rules, regulatory rules, and payer-specific rules. The structural rules stored in the rules database 122 may define procedure-location validity constraints (e.g., that a particular procedure may be permissible only in a designated treatment location, such as a gastrointestinal (GI) treatment room) and inter-procedure dependency constraints. The regulatory rules stored in the rules database 122 may include CMS rules, AMA rules, state-specific rules, and payer-specific rules. The rules database 122 may be communicatively coupled to the external coding standards 134 data source, such that updates to medical coding standards detected in external databases may be used to update the predefined coding rules stored in the rules database 122.

The reinforcement learning module 124 may execute operations to dynamically adjust the weight score for validated candidate code pairs in the PRP matrix 114 based on a reward signal. The reinforcement learning module 124 may execute a multi-armed bandit algorithm, using different code combinations as arms with varying reward levels. The multi-armed bandit algorithm executed by the reinforcement learning module 124 may be a contextual bandit algorithm. The contextual features may define the state space and may comprise the procedure code, the payer identifier, the location identifier, the patient class, and a service date range. The reinforcement learning module 124 may derive the reward signal from downstream outcome data by computing a composite function of an approval status and a transaction outcome (e.g., reimbursement outcome) associated with processed transactional records. For example, downstream outcome data may include the approval status and transaction outcome for processed transactional records.

The reward signal computed by the reinforcement learning module 124 may be a composite function comprising a reimbursement component weighted by a first coefficient (α), an approval-indicator component weighted by a second coefficient (β), and a constraint-violation penalty component weighted by a third coefficient (γ). The reward signal may be defined as

$$R = α * ( \frac{reimbursement received}{expected reimbursement} ) + β * ( approval indicator ) - ( γ * ( compliance penalty ) ) \qquad ( 1 )$$

where α, β, and γ may be configurable coefficients initialized from hierarchical contract rules, with initial values such as α=0.6, β=0.3, and γ=0.1. The reinforcement learning module 124 may produce an adjusted weight score for each validated candidate code pair based on the reward signal. The reinforcement learning module 124 may update the weight score for each candidate code pair using an incremental averaging rule, defined by the equation

Q_{new(a)}=Q_{current}(a)+(1/N(a))*(R−Q_{current(a)}) (2)
where ‘Q(a)’ may be the estimated reward for action a, ‘N(a)’ may be the number of times action a has been selected, and R may be the observed reward signal. The reinforcement learning module 124 may balance the exploration of new code combinations with the exploitation of known successful combinations by calculating an exploration-exploitation score for each validated candidate code pair using an upper confidence bound (UCB) algorithm, the exploration-exploitation score balancing an estimated cumulative reward for the validated candidate code pair against a degree of uncertainty derived from a ratio of a total count of selections across all validated candidate code pairs to a count of times the validated candidate code pair has been selected. The UCB algorithm may select the action maximizing using the equation:

$$UCB\_n ( a ) = \hat{Q} ( a ) + c * \sqrt{\frac{ln ( n )}{N ( a )}} . \qquad ( 3 )$$

where \hat{Q}(a) may be the estimated reward for action a, n may be the total number of selections, N(a) may be the number of times action a has been selected, and c may be an exploration parameter.

The reinforcement learning module 124 may execute operations to update or modify the weight score for the validated candidate code pair by dynamically or incrementally adjusting the weight score toward the reward signal in proportion to an inverse of the count of times the validated candidate code pair has been selected. The reinforcement learning module 124 may execute operations in real-time, recalculating based on the entire historical data as new data and decisions are inserted, and may provide the latest decision outputs. The reinforcement learning module 124 may receive relevance feedback from a domain analyst via the user interface/analyst review module 132. The relevance feedback may include an acceptance, a rejection, or a modification of a validated candidate code pair, and may incorporate the relevance feedback as a ground truth label into the reward signal to update the weight score in the PRP matrix 114.

The audit trail database 126 may be configured to store an audit record for each code combination recommendation generated by the reinforcement-learning-driven profile matrix optimization system 110. Each audit record stored in the audit trail database 126 may comprise an original code assignment of the target transactional record, a recommended code pair corresponding to the validated candidate code pair, a confidence score, a model version identifier, and a timestamp. The audit trail database 126 may be implemented as a tamper-resistant audit data store with encryption at rest and in transit, role-based access controls, and logging of all data access and modifications. The audit trail database 126 may be accessible through a separate interface for compliance review and quality assurance purposes, enabling authorized personnel to review the decision-making process behind code selections.

The sandbox testing module 128 may execute operations to simulate sandbox testing by applying validated candidate code pairs to a selected subset of historical transactional records. The sandbox testing module 128 may execute operations as a parallel instance of the production system operating on the most recent, for example, six months of historical claims, ranked by payer, procedure type, and location. The sandbox testing module 128 may compare the predicted outcome value determined for each validated candidate code pair against an actual transaction outcome value associated with a corresponding historical transactional record in the selected subset. The sandbox testing module 128 may apply an acceptance threshold requiring that predicted reimbursement may be in, for example, ±5% of actual reimbursement for more than 90% of test claims. In response to detection of a deviation between the predicted outcome value and the actual transaction outcome value exceeding the predefined acceptance threshold for more than a predefined proportion of historical transactional records in the selected subset, the sandbox testing module 128 may retrieve a prior version of the PRP matrix 114 via a version control mechanism and may replace the PRP matrix 114 with the prior version to maintain system stability. The sandbox testing module 128 may also implement A/B testing that compares a new model version against a current production version, with automatic rollback if the new version underperforms on any measured metric by more than 2%.

The ranked code recommendations 130 may represent the output of the combinatorial claims optimizer engine 118. The ranked code recommendations 130 may include a set of recommended code combinations that have been validated by the code combination validation module 116, rated by the iPRC 120, and ranked by expected transaction outcome values. The ranked code recommendations 130 may be prioritized based on their potential for maximizing reimbursement while maintaining compliance with coding regulations. The ranked code recommendations 130 may be presented to the user interface/analyst review module 132 for human assessment.

The user interface/analyst review module 132 may execute operations to render, display, or present the ranked code recommendations 130 to revenue cycle analysts or medical coders. The user interface/analyst review module 132 may be integrated into a hospital's existing billing software, enabling users to review and apply recommendations without switching between multiple applications. The user interface/analyst review module 132 may provide a side-by-side comparison of original codes and recommended optimized combinations, along with estimated reimbursement differences and compliance indicators. In an embodiment, the user interface/analyst review module 132 may be implemented, for example, as a PowerApps action tracker that provides a review queue where analysts may accept, reject, or modify recommendations. The user interface/analyst review module 132 may be communicatively coupled to the reinforcement learning module 124, and analyst decisions captured through the user interface/analyst review module 132 may feed back into the reinforcement learning module 124 as ground truth labels, forming a closed-loop feedback mechanism between human review and algorithmic weight adjustment. The user interface/analyst review module 132 may render or display each flagged-for-manual-review candidate code pair with the confidence score, the original code assignment, and the predicted outcome value, and an analyst action on the user interface/analyst review module 132 may be recorded as a ground truth label.

The external coding standards 134 may represent external data sources from which updates to medical coding standards may be received by the reinforcement-learning-driven profile matrix optimization system 110. The external coding standards 134 may include data feeds from the CMS, the AMA, and state Medicaid portals. In an embodiment, the reinforcement-learning-driven profile matrix optimization system 110 may monitor the external coding standards 134 via an application programming interface (API), and monitoring the external coding standards 134 may comprise executing a natural language processing (NLP) pipeline comprising named entity recognition (NER) and text classification to parse unstructured text of updated coding manuals received from the external coding standards 134. The NLP pipeline may extract modified rule parameters from the unstructured text, and the extracted parameters may be dynamically mapped to the rules database 122 without disrupting ongoing claims processing by the combinatorial claims optimizer engine 118.

In another embodiment, monitoring of the external coding standards 134 may be performed by parsing structured update feeds from the CMS and the AMA databases using pattern-matching and field-extraction rules. The reinforcement-learning-driven profile matrix optimization system 110 may revalidate existing procedure-revenue code combinations stored in the PRP matrix 114 against the updated rules following an update to the rules database 122 from the external coding standards 134.

The rate cards/contracts 136 may represent the source of payer contract rate data used by the iPRC 120. The rate cards/contracts 136 may include rate card records, including effective dates, effective start dates, plan identifiers, contracted rates, payment methodology designations, and associated weights. The rate cards/contracts 136 may supply rate data from different locations and may include data elements such as revenue codes, procedure codes, fee schedule (FS) codes, diagnosis-related group (DRG) codes, DRG weights, and plan values that feed into the hierarchical calculation engine of the iPRC 120.

The reinforcement-learning-driven profile matrix optimization system 110 may execute operations automatically or dynamically transmit a structured data output. For example, the structured data output may include the validated candidate code pair, the predicted outcome value, a confidence score derived from the adjusted weight score, and a model version identifier, and send them to an external data system (e.g., healthcare-related data). The structured data output may be integrated into the target transactional record via an API coupled to the distributed databases in which the PRP matrix 114, the rules database 122, and the audit trail database 126 may be stored.

In operation, the reinforcement-learning-driven profile matrix optimization system 110 may receive historical transactional records from the hospital billing system 102, the hospital billing system 2 104, and the hospital billing system N 106 through the data extraction and loading module 108, which may normalize heterogeneous data from these disparate systems into a standardized format stored in columnar storage partitions of distributed databases. The standardized records may be processed by the machine learning engines 112, which may apply principal component analysis to project the historical transactional records into an orthogonal, lower-dimensional feature space for each partition defined by the payer identifier and the location identifier, and may execute association rule mining and integer programming to construct and populate the PRP matrix 114 with statistically significant procedure-revenue code pairings indexed by payer and location. The machine learning engines 112 may identify procedure codes having a mean transaction outcome value (e.g., reimbursement value) exceeding zero across records associated with non-zero transaction outcome values, and may map a usage metric and a weight score to each corresponding cell of the PRP matrix 114. When a new medical claim is received, the combinatorial claims optimizer engine 118 may retrieve applicable candidate code pairs from the PRP matrix 114 and generate multiple valid procedure-revenue code combinations. The code combination validation module 116 may validate these candidate code pairs against structural rules and regulatory rules stored in the rules database 122, may apply the location-constraint checker and the dependency-constraint checker to verify permissibility and inter-procedure compatibility, and may filter out non-compliant combinations. The code combination validation module 116 may classify each remaining validated candidate code pair as auto-approved, auto-rejected, or flagged-for-manual-review based on a confidence score derived from the weight score and the usage metric.

The iPRC 120 may calculate a predicted outcome value (e.g., expected reimbursement output) for each validated candidate code pair by selecting the applicable rate record from the rate cards/contracts 136 based on temporal priority and the specificity hierarchy prioritizing plan-specific over location-specific over general rate records, and by applying the rate formula corresponding to the payment methodology designated for the payer-plan-location combination. The rate exception engine of the iPRC 120 may evaluate applicable exception rules from among the plurality of health plan rules stored in the rate data store. The reinforcement learning module 124 may dynamically adjust the weight scores in the PRP matrix 114 based on a reward signal derived from downstream outcome data, the downstream outcome data comprising an approval status and a transaction outcome (e.g., reimbursement outcome) associated with processed transactional records, using the contextual bandit algorithm with the UCB exploration-exploitation score to balance exploration of new code combinations with exploitation of historically successful combinations, and may produce an adjusted weight score for each validated candidate code pair. The sandbox testing module 128 may validate the performance of updated models and matrix versions against a ranked subset of historical claims before deployment, may compare predicted outcome values against actual transaction outcome values, and may retrieve a prior version of the PRP matrix 114 via a version control mechanism in response to deviations exceeding the predefined acceptance threshold. The ranked code recommendations 130, comprising validated and rated code combinations, may be presented through the user interface/analyst review module 132 to revenue cycle analysts, whose acceptance, rejection, or modification decisions may be captured as relevance feedback and included as ground truth labels into the reward signal of the reinforcement learning module 124 to update the weight scores in the PRP matrix 114.

The audit trail database 126 may record each recommendation as a tamper-resistant audit record comprising the original code assignment, the recommended code pair, the confidence score, the model version identifier, and the timestamp, for compliance review and quality assurance. The reinforcement-learning-driven profile matrix optimization system 110 may thereby transform the computationally intractable combinatorial search space of candidate procedure-revenue code pairs into a tractable, weighted, and continuously refined matrix, and may automatically transmit a structured data output comprising the validated candidate code pair, the predicted outcome value, a confidence score derived from the adjusted weight score, and a model version identifier to an external data system for integration into the target transactional record via an API coupled to the distributed databases.

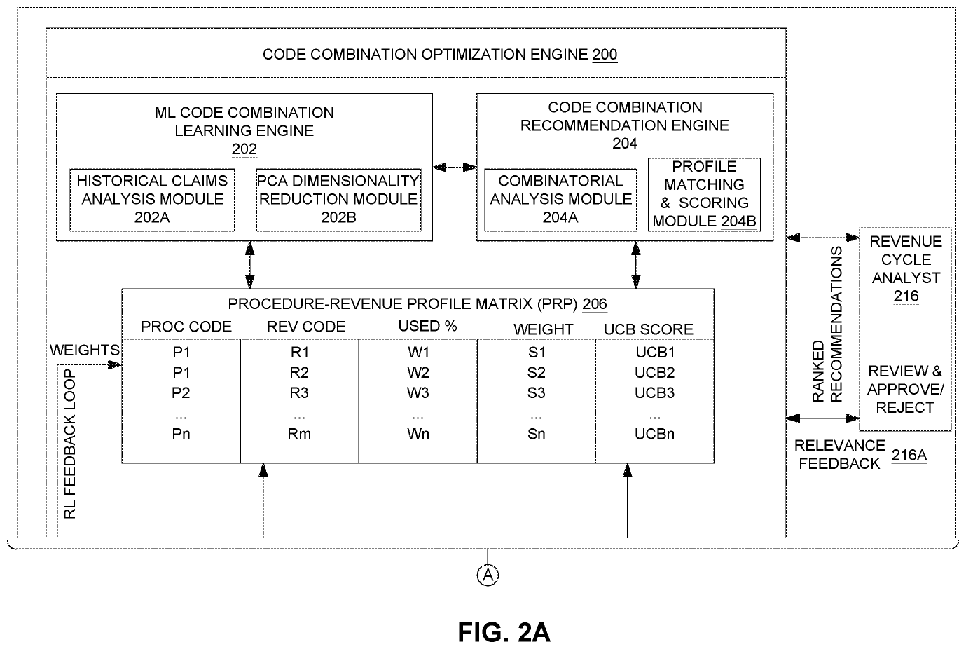

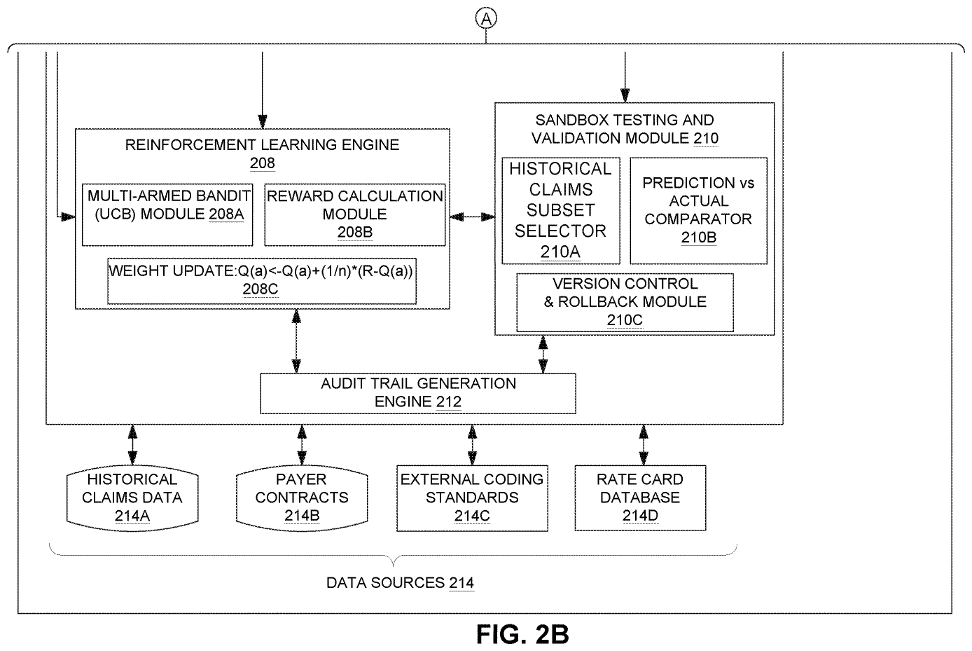

FIG. 2A and FIG. 2B illustrates a code combination optimization engine 200, according to an exemplary embodiment. FIG. 2A and FIG. 2B is described in conjunction with FIG. 1. 2A shows an initial portion of the code combination optimization engine 200, which includes an ML code combination learning engine 202, a code combination recommendation engine 204, a procedure-revenue profile (PRP) matrix 206, and a revenue cycle analyst 216. FIG. 2B shows the next portion of the code combination optimization engine 200 that includes a reinforcement learning engine 208, a sandbox testing and validation module 210, an audit trail generation engine 212, and data sources 214. In an embodiment, FIG. 2A and FIG. 2B describes the internal architecture of the code combination optimization engine 200, the data structures maintained therein, the feedback pathways connecting the revenue cycle analyst 216 to the reinforcement learning engine 208, and the data sources 214 from which the code combination optimization engine 200 may derive its operating parameters.

Referring to FIG. 2A, the ML code combination learning engine 202 may be a machine learning engine that may execute operations to receive multiple historical transactional records extracted and loaded by the data extraction and loading module 108 of FIG. 1. The ML code combination learning engine 202 may include a historical claims analysis module 202A and a principal component analysis (PCA) dimensionality reduction module 202B. The historical claims analysis module 202A may execute operations to receive historical transactional records from the data sources 214 (shown in FIG. 2B). Each historical transactional record may include at least a procedure code, a revenue code, a payer identifier, and a transaction outcome value in a standardized format stored in one or more columnar storage partitions of a distributed database. The historical claims analysis module 202A may process correlations between procedure codes and revenue codes in the historical transactional records by applying association rule mining to identify statistical pairings of procedure codes and revenue codes across different payer identifiers and location identifiers. The historical claims analysis module 202A may execute operations to filter the historical transactional records and retain only records associated with non-zero transaction outcome values. The historical claims analysis module 202A may eliminate pairings of procedure codes and revenue codes in which the mean transaction outcome value across records with non-zero transaction outcome values does not exceed zero. The historical claims analysis module 202A may eliminate each pairing that may be associated with fewer than a minimum sample count of records, where the minimum sample count may be auto-calculated by the ML code combination learning engine 202 to eliminate pairings supported by fewer than three sample data points.

The PCA dimensionality reduction module 202B may execute operations to project multiple historical transactional records into a standardized format into an orthogonal, lower-dimensional feature space. Upon projection, the PCA dimensionality reduction module 202B may generate multiple principal components satisfying a predetermined variance threshold. The predetermined variance threshold may be, for example, approximately 95%, and the PCA dimensionality reduction module 202B may retain between 10 and 12 principal components based on the features present in the historical transactional records. The input features subjected to dimensionality reduction by the PCA dimensionality reduction module 202B may include payer identifier, benefit plan, location, patient class, contract period, service date, admission date, diagnosis-related group (DRG) code, and procedure code. The PCA dimensionality reduction module 202B may apply the projection into the orthogonal, lower-dimensional feature space for each partition of the multiple historical transactional records defined by the payer identifier and the location identifier, thereby generating payer-specific and location-specific principal components. The PCA dimensionality reduction module 202B may receive historical transactional records from the historical claims analysis module 202A and may output the multiple principal components to the code combination recommendation engine 204 and to the PRP matrix 206. The architecture of the PCA dimensionality reduction module 202B may be scalable to accommodate additional variables as new payer contracts and business requirements are introduced into the reinforcement-learning-driven profile matrix optimization system 110.

The code combination recommendation engine 204 may be implemented as a component of the machine learning engines 112 and may include a combinatorial analysis module 204A and a profile matching and scoring module 204B. The combinatorial analysis module 204A may execute operations in integer programming to combinatorially optimize candidate code pairs derived from the PRP matrix 206. The combinatorial analysis module 204A may evaluate permutations of procedure codes and revenue codes, taking into account payer-specific policies, location-specific rules, and historical reimbursement data stored in the PRP matrix 206. The combinatorial analysis module 204A may identify or determine candidate code pairs that may maximize a predicted outcome value while maintaining compliance with the structural rule and the regulatory rule from the rules database 122 of FIG. 1. The profile matching and scoring module 204B may execute operations to receive a target transactional record and to match the target transactional record against profiles stored in the PRP matrix 206 based on a combination of the payer identifier, a plan identifier, and a location identifier associated with the target transactional record. The profile matching and scoring module 204B may score each candidate code pair by deriving a confidence score from the weight score and the usage metric stored in the PRP matrix 206.

The PRP matrix 206 may be a partitioned data structure stored in one or more columnar storage partitions of the distributed database and may be indexed for retrieval by a combination of procedure code, revenue code, and payer identifier. As shown in FIG. 2A, the PRP matrix 206 may include columns designated as PROC CODE, REV CODE, WEIGHT, UCB SCORE, and USED %. The PROC CODE column may store a plurality of procedure codes designated P1, P2, through Pn. The REV CODE column may store a plurality of revenue codes designated R1, R2, R3, through Rm, where each revenue code may be mapped to at least one procedure code. The WEIGHT column may store weight scores designated W1, W2, W3, through Wn, where each weight score may correspond to a cell defined by a specific pairing of a procedure code and a revenue code. The UCB SCORE column may store exploration-exploitation scores designated S1, S2, S3, through Sn, where each exploration-exploitation score may be computed by the reinforcement learning engine 208 (shown in FIG. 2B) for each candidate code pair. The USED % column may store usage metrics designated U1, U2, U3, through Un, where each usage metric represents a percentage calculated as the count of approved claims associated with a given procedure code and revenue code combination divided by the total count of approved claims associated with that procedure code. The PRP matrix 206 may be partitioned by the payer identifier such that a given procedure code may have a different weight score for a first payer identifier than for a second payer identifier. The PRP matrix 206 may further store an effective date and a status indicator for each candidate code pair.

A revenue cycle analyst 216 may be presented with ranked recommendations generated by the code combination recommendation engine 204 through the user interface/analyst review 132 of FIG. 1. The revenue cycle analyst 216 may review and approve or reject the ranked recommendations. The revenue cycle analyst 216 may provide relevance feedback 216A, which may be fed back into the code combination optimization engine 200 via a reinforcement learning (RL) feedback loop. The relevance feedback 216A provided by the revenue cycle analyst 216 may serve as ground-truth labels for the reinforcement learning engine 208. The analyst actions recorded in the user interface/analyst review 132 may be stored as training signals used by the reinforcement learning engine 208 to dynamically adjust the weight scores in the PRP matrix 206. The code combination recommendation engine 204 may classify each candidate code pair into one of the auto-approved, the auto-rejected, or flagged-for-manual-review based on the confidence score derived from the weight score and the usage metric, and the revenue cycle analyst 216 may act upon those candidate code pairs classified as flagged-for-manual-review.

Referring to FIG. 2B, the reinforcement learning engine 208 may include a multi-armed bandit (UCB1) module 208A, a reward calculation module 208B, and a weight update module 208C. The multi-armed bandit (UCB) module 208A may execute operations to implement a contextual bandit algorithm, in which different candidate code pairs may be treated as arms with varying reward levels. The multi-armed bandit (UCB) module 208A may calculate an exploration-exploitation score for each validated candidate code pair by balancing an estimated cumulative reward for the validated candidate code pair against a degree of uncertainty derived from a ratio of a total count of selections across all validated candidate code pairs to a count of times the validated candidate code pair has been selected. The state space of the multi-armed bandit (UCB) module 208A may include procedure code, payer identifier, location, patient class, and service date range as contextual features defining the clinical and administrative context of a claim. The action space of the multi-armed bandit (UCB1) module 208A may include the set of valid revenue codes for a given procedure code, as determined by the PRP matrix 206.

The reward calculation module 208B may execute operations to derive a reward signal from downstream outcome data by computing a composite function of an approval status and a transaction outcome associated with one or more processed transactional records. The reward signal may be a composite function comprising a reimbursement component weighted by a first coefficient, an approval-indicator component weighted by a second coefficient, and a constraint-violation penalty component weighted by a third coefficient. The weights applied by the reward calculation module 208B may be initialized from hierarchical contract rules stored in the rate cards/contracts 136 of FIG. 1.

The weight update module 208C may execute operations to update the weight score for each validated candidate code pair by incrementally adjusting the weight score toward the reward signal in proportion to an inverse of the count of times the validated candidate code pair has been selected. As shown in FIG. 2B, the weight update module 208C may apply the update rule

Q(a)←Q(a)+1/n*(R−Q(a)) (4)
where ‘Q(a)’ may represent the estimated reward for a given candidate code pair designated action a, ‘n’ may represent the number of times the given candidate code pair has been selected, and ‘R’ may represent the observed reward signal computed by the reward calculation module 208B. The weight update module 208C may write the adjusted weight scores back to the WEIGHT column and the adjusted exploration-exploitation scores back to the UCB SCORE column of the PRP matrix 206. The reinforcement learning engine 208 may perform continuous updates, recalculating based on the entire historical dataset as new transaction data and analyst decisions may be inserted into the data sources 214.

The sandbox testing and validation module 210 may include a historical claims subset selector 210A, a prediction versus actual comparator 210B, and a version control and rollback module 210C. The historical claims subset selector 210A may execute operations to select a subset of multiple historical transactional records from the historical claims data 214A of the data sources 214. The subset may be ranked by payer identifier, procedure type, and location, and may represent the most recent six months of historical transactional records. The prediction versus actual comparator 210B may execute operations to apply validated candidate code pairs to the ranked subset selected by the historical claims subset selector 210A. The prediction versus actual comparator 210B may compare the predicted outcome value determined for each validated candidate code pair against the actual transaction outcome value associated with a corresponding historical transactional record in the ranked subset. In response to the detection of a deviation between the predicted outcome value and the actual transaction outcome value exceeding a predefined acceptance threshold for more than a predefined proportion of historical transactional records in the ranked subset, the version control and rollback module 210C may retrieve a prior version of the PRP matrix 206 and may replace the current PRP matrix 206 with the prior version. The predefined acceptance threshold may be set at plus or minus five percent, and the predefined proportion may be set at greater than ninety percent of test claims. The version control and rollback module 210C may automatically roll back to a prior version if a new version underperforms any measured metric by more than 2% relative to the current production version.

The audit trail generation engine 212 may execute operations to generate and store an audit record for each validated candidate code pair in the audit trail database 126. Each audit record may include an original code assignment of the target transactional record, a recommended code pair corresponding to the validated candidate code pair, a confidence score, a model version identifier, and a timestamp. The audit trail generation engine 212 may store the audit records in a tamper-resistant audit data store, and the audit records may be accessible through the user interface/analyst review 132 for compliance review purposes.

The data sources 214 may include historical claims data 214A, payer contracts 214B, external coding standards 214C, and a rate card database 214D. The historical claims data 214A may correspond to the historical transactional records extracted from the healthcare data systems 102, 104, 106 by the data extraction and loading module 108. The payer contracts 214B may store payer-specific contract terms and reimbursement schedules. The external coding standards 214C may store coding rules and regulatory guidelines from external regulatory bodies. The rate card database 214D may store rate records indexed by effective date and organized according to a priority hierarchy that may prioritize a plan-specific rate record over a location-specific rate record over a general rate record. The data sources 214 may provide input data to the ML code combination learning engine 202, the reinforcement learning engine 208, and the sandbox testing and validation module 210.

In operation, the code combination optimization engine 200 may receive historical transactional records from the data sources 214 via the data extraction and loading module 108 of FIG. 1. The historical claims analysis module 202A may process the historical transactional records to identify pairings of procedure codes and revenue codes having a mean transaction outcome value exceeding zero and supported by at least the auto-calculated minimum sample count. The PCA dimensionality reduction module 202B may then project the historical transactional records into the orthogonal, lower-dimensional feature space on a per-payer and per-location basis, retaining principal components satisfying the 95% variance threshold. The ML code combination learning engine 202, executing association rule mining on the plurality of principal components, may construct the PRP matrix 206 by mapping a usage metric and a weight score to each cell indexed by procedure code, revenue code, and payer identifier. When a target transactional record may be received for processing, the profile matching and scoring module 204B of the code combination recommendation engine 204 may match the target transactional record against profiles in the PRP matrix 206 based on the payer identifier, plan identifier, and location identifier. The combinatorial analysis module 204A may then execute integer programming to identify candidate code pairs that may maximize the predicted outcome value, subject to the structural and regulatory rules stored in the rules database 122. The validated candidate code pairs may be ranked and transmitted to the revenue cycle analyst 216 for review through the user interface/analyst review 132. The revenue cycle analyst 216 may approve, reject, or modify the ranked recommendations, and the relevance feedback 216A may be fed back through the RL feedback loop to the reinforcement learning engine 208 shown in FIG. 2B. The multi-armed bandit (UCB) module 208A may compute the exploration-exploitation score for each candidate code pair, the reward calculation module 208B may compute the composite reward signal from the downstream outcome data, and the weight update module 208C may apply the incremental update rule as defined in equation (4) above to adjust the weight scores in the PRP matrix 206. The sandbox testing and validation module 210 may periodically validate updated versions of the PRP matrix 206 by applying validated candidate code pairs to the ranked subset selected by the historical claims subset selector 210A, comparing predictions against actual transaction outcomes via the prediction versus actual comparator 210B, and rolling back to a prior version via the version control and rollback module 210C where deviations may exceed the predefined acceptance threshold. The audit trail generation engine 212 may record each recommendation, analyst action, and model version in the audit trail database 126, thereby maintaining a traceable record of the operations performed by the code combination optimization engine 200.

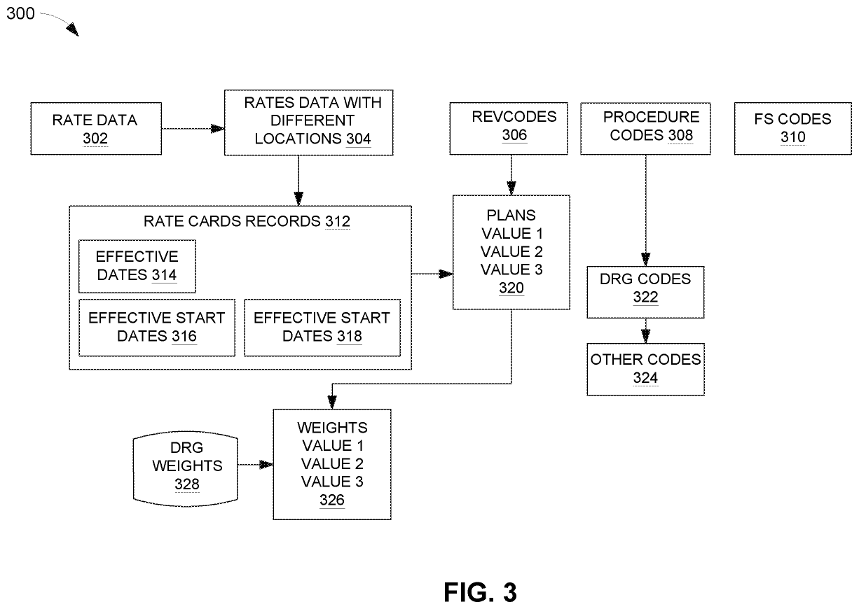

FIG. 3 illustrates a block diagram 300 showing a rate card and plans data structure, according to an exemplary embodiment. FIG. 3 is described in conjunction with FIG. 1 and FIGS. 2A-2B. In an embodiment, FIG. 3 illustrates the rate card and plans data structure which may include rate data 302 component, rates data with different locations 304 component, revenue codes (RevCodes) 306 component, procedure codes 308 component, fee schedule (FS) codes 310 component, rate cards records 312 component, effective dates 314 component, effective start dates 316 component, effective start dates 318 component, plans 320 component, DRG codes 322 component, other codes 324 component, weights 326 component, and DRG weights 328 component.

The rate data 302 may be received from healthcare data systems via the data extraction and loading module 108. The rate data 302 may be organized by service location and may flow into the rate data with different locations 304. The rates data with different locations 304 may store location-differentiated rate records such that, for a given procedure code and revenue code pairing, a separate rate amount may be maintained for each service location associated with a payer contract. The location-specific segmentation in the rates data across different locations 304 may enable a priority hierarchy in which a plan-specific rate record may be prioritized over a location-specific rate record, and a location-specific rate record may be prioritized over a general rate record. The rates data with different locations 304 may be coupled to the RevCodes 306, the procedure codes 308, and the FS codes 310. The RevCodes 306 may store the set of revenue codes associated with each payer contract. The procedure codes 308 may store the procedure codes corresponding to medical services covered by each rate card. The FS codes 310 may store fee schedule rate values indexed by the procedure code, the service location, and the effective date, which may be applied by the iPRC 120 when processing a target transactional record against the rate card and plans data structure 300. The RevCodes 306, the procedure codes 308, and the FS codes 310 may each be represented as multi-instance data stores to accommodate the multiplicity of codes arising from different payer contracts and service locations.

The rate cards records 312 may store structured rate card data organized by effective dates 314. The effective dates 314 may include, for example, a first set of effective start dates 316 and a second set of effective start dates 318. The first set of effective start dates, 316, and the second set of effective start dates, 318, may represent temporal boundaries of rate card applicability. The iPRC 120 may select the appropriate rate record by matching the service date of the target transactional record against the effective start dates 316 and the effective start dates 318 stored in the rate cards records 312. Where a payer contract undergoes a rate change at a particular date, the rate cards records 312 may retain both the prior effective start dates 316 and the subsequent effective start dates 318, thereby enabling the temporal attribute-based selection of the rate record recited in the claims. The rate cards records 312 may be coupled to the rates data with different locations 304 via a directional data connection, such that the rate card effective date range may be resolved in conjunction with the service location for each target transactional record.

The plans 320 component may store plan-level parameters, represented by values 1, 2, and 3. The plans 320 may correspond to different insurance plan types, benefit plan configurations, or billing structures maintained in a given payer contract. The plans 320 may receive data connections from the procedure codes 308 and from the RevCodes 306, indicating that the plan-level parameters stored in the plans 320 may define plan-specific rate applicability for each procedure code and revenue code combination. The plan-level segmentation in the plans 320 may enable the iPRC 120 to apply a rate formula selected based on a combination of the payer identifier, the plan identifier, and the location identifier associated with the target transactional record. In an embodiment, the plans 320 may accommodate multiple payment mechanisms, where value 1, value 2, and value 3 stored in the plans 320 may represent parameters associated with different rate formula types, such as a per-case formula, a per-diem formula, a diagnosis-group-based formula, a percentage-of-charges formula, a per-unit formula, and a fee-schedule formula.

The DRG codes 322 may store diagnosis-related group classification codes used for inpatient claims processing under payment systems such as the acute care hospital inpatient prospective payment system (IPPS). The DRG codes 322 may receive data connections from the procedure codes 308 and may feed data connections into both the plans 320 and the weights 326, indicating that the DRG classification may influence both the plan-level rate selection and the weight-based reimbursement calculation. The other codes 324 may store additional code types beyond procedure codes, revenue codes, DRG codes, and fee schedule codes, and may be connected to the DRG codes 322 via a bidirectional data path. In an embodiment, the other codes 324 may include grouping scheme codes, ambulatory payment classification (APC) codes for outpatient services, or other payer-specific code classifications.

The weights 326 may store weight values, represented as value 1, value 2, and value 3, that may be applied in reimbursement calculations performed by the iPRC 120 of FIG. 1. The weights 326 may receive data connections from the rate cards records 312 and from the DRG codes 322, and a bidirectional data connection may be maintained between the weights 326 and the other codes 324. The DRG weights 328 may be a subset of the weights 326 and may be stored in a database accessed via structured query language (SQL). The DRG weights 328 may store numerical weight values that correspond to the relative resource consumption and clinical complexity associated with each DRG classification. The DRG weights 328 may enable the diagnosis-group-based formula, where a payment amount may be computed as a base rate multiplied by the DRG weight value stored in the DRG weights 328 and further multiplied by a location adjustment factor derived from the rates data for different locations 304. The data connection from the DRG weights 328 to the rate cards records 312 may indicate that the weight values are temporally bounded by the effective dates 314, such that rate changes to DRG weight assignments are reflected in the rate card and plans data structure 300 without requiring modification to the underlying calculation logic.

In an embodiment, the rate card and plans data structure may facilitate the evaluation of exception rules spanning the plans 320, the rates data with different locations 304, and the rate cards records 312, as performed by the rate exception engine of the iPRC 120. The rate card and plans data structure 300 may store parameters for, for example, 772,546 or more health plan rules spanning 71 or more plans, 12 or more service locations, and 39 or more contracts, such that the rate exception engine may evaluate which exception rules apply to a given target transactional record by traversing the interconnected data paths between the plans 320, the rates data with different locations 304, the rate cards records 312, and the weights 326. In various embodiments, the rate exception engine may process at least 100,000, at least 250,000, at least 500,000, or at least 750,000 health plan rules. In various embodiments, the rate exception engine may process rules across at least 20, at least 40, at least 60, or at least 70 plans. In various embodiments, the rate exception engine may process rules across at least 10, at least 20, at least 30, or at least 40 contracts.

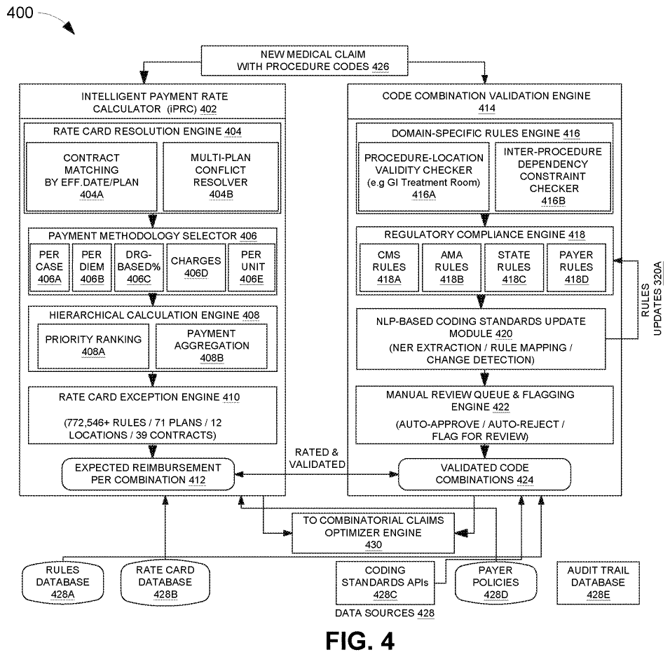

FIG. 4 illustrates a block diagram 400 showing a medical code combination optimization system, according to an exemplary embodiment. FIG. 4 is described in conjunction with FIGS. 1-3. In an embodiment, FIG. 4 illustrates the block diagram 400 of the medical code combination optimization system. The block diagram 400 shows an iPRC 402 component, a code combination validation engine 414, a data sources block 428, a new medical claim with procedure codes input 426, a validated code combinations output 424, and an output path to a combinatorial claims optimizer engine 430. The iPRC 402 may include a rate card resolution engine 404, a payment methodology selector 406, a hierarchical calculation engine 408, a rate card exception engine 410, and an expected reimbursement per combination output 412. The code combination validation engine 414 may include a domain-specific rules engine 416, a regulatory compliance engine 418, a natural language processing (NLP)-based coding standards update module 420, and a manual review queue and flagging engine 422. The data sources block 428 may include a rules database 428A, a rate card database 428B, rated-and-validated coding standards application programming interfaces (APIs) 428C, payer policies 428D, and an audit trail database 428E.

The iPRC 402 may execute operations to determine the predicted outcome value for each validated candidate code pair received from the PRP matrix 206, as described with reference to FIG. 2A and FIG. 2B. The iPRC 402 may receive, as input, the rate card data, the claim data described, and the candidate code pairs generated by the machine learning engines. The iPRC 402 may process these inputs through the rate card resolution engine 404, the payment methodology selector 406, the hierarchical calculation engine 408, and the rate card exception engine 410 to produce the expected reimbursement per combination 412.

The rate card resolution engine 404 may execute operations to resolve which rate record from the rate card database 428B applies to a given target transactional record. The rate card resolution engine 404 may include a contract matching by effective date and plan submodule 404A, and a multi-plan conflict resolver submodule 404B. The contract matching by effective date and plan submodule 404A may execute operations to select a rate record from the rate card database 428B based on a temporal attribute of the target transactional record. The temporal attribute may be the service date or the admission date associated with the target transactional record. The contract matching by effective date and plan submodule 404A may compare the service date of the target transactional record against the effective start dates and effective end dates stored in the rate cards records, and may select the rate record whose effective date range includes the service date. The multi-plan conflict resolver submodule 404B may execute operations to resolve conflicts when multiple rate records from different plans or contracts may apply to the same target transactional record. The multi-plan conflict resolver submodule 404B may apply a priority hierarchy in which a plan-specific rate record may be prioritized over a location-specific rate record, and a location-specific rate record may be prioritized over a general rate record. The priority hierarchy applied by the multi-plan conflict resolver submodule 404B may correspond to the hierarchical calculation.

The payment methodology selector 406 may execute operations to select a rate formula based on a combination of the payer identifier, a plan identifier, and a location identifier associated with the target transactional record. The payment methodology selector 406 may include a per case submodule 406A, a per diem submodule 406B, a diagnosis-related group (DRG)-based percentage submodule 406C, a charges submodule 406D, and a per unit submodule 406E. The per case submodule 406A may execute operations to calculate a payment using a formula in which a base rate may be multiplied by a DRG weight and a location adjustment factor, for example, Payment=BaseRate*DRG_Weight*LocationAdjustment. The per diem submodule 406B may execute operations to calculate a payment using a formula in which a daily rate is multiplied by the length of stay, capped at a contractual maximum, for example, Payment=DailyRate*LengthOfStay. The computed value may be capped at a contractual maximum if the computed value exceeds the contractual maximum. The DRG-based percentage submodule 406C may execute operations to calculate a payment based on a percentage applied to a DRG-derived base amount. The charges submodule 406D may execute operations to calculate a payment using a percentage-of-charges formula, in which the total charge amount is multiplied by a contracted percentage, expressed as, for example, Payment=TotalCharges*ContractedPercentage. The per unit submodule 406E may execute operations to calculate a payment using a per-unit aggregate formula in which a unit rate for each line item may be multiplied by the number of units for that line item, and the products may be summed across all line items, expressed as

Payment=ΣUnitRateᵢ*Units_i (5)
for each line item i.

In another embodiment, the payment methodology selector 406 may further support a fee-schedule formula in which a fee schedule rate may be looked up based on the procedure code, the location, and the effective date. The payment methodology selector 406 may determine which of the per case submodule 406A, the per diem submodule 406B, the DRG-based percentage submodule 406C, the charges submodule 406D, the per unit submodule 406E, or the fee-schedule formula may be applied based on the payer-plan-location combination identified for the target transactional record.

The hierarchical calculation engine 408 may execute operations to apply the selected rate formula to the rate record selected by the rate card resolution engine 404. The hierarchical calculation engine 408 may include a priority ranking submodule 408A and a payment aggregation submodule 408B. The priority ranking submodule 408A may execute operations to rank and sequence the calculation steps according to the priority hierarchy described above, in which plan-specific rates may be applied before location-specific rates, and location-specific rates may be applied before general rates. The payment aggregation submodule 408B may execute operations to aggregate the individual payment computations across all line items and all applicable rate formulas for a given target transactional record into a single predicted outcome value. The hierarchical calculation engine 408 may process data from the rate card and plans data structure 300. The plans component, the weights component, the DRG weights, and the effective dates may be used to parameterize the rate formulas selected by the payment methodology selector 406.

The rate card exception engine 410 may execute operations to evaluate a set of exception rules spanning a set of plans, a set of service locations, and a set of contracts stored in the rate card database 428B. The rate card exception engine 410 may process 772,546 or more health plan rules across 71 plans, 12 locations, and 39 contracts. The rate card exception engine 410 may identify rate exceptions where the standard rate formula may not apply due to contract-specific overrides, carve-outs, or special pricing arrangements. When the rate card exception engine 410 identifies an applicable exception rule, the rate card exception engine 410 may override the standard rate calculation performed by the hierarchical calculation engine 408 with the exception-specified rate. The output of the iPRC 402 may be the expected reimbursement per combination 412, representing the computed expected payment for each candidate code pair evaluated by the iPRC 402.

The code combination validation engine 414 may execute operations to validate candidate code pairs from the procedure-revenue profile matrix against structural rules and regulatory rules stored in the rules database 428A. The code combination validation engine 414 may receive, as input, a new medical claim with procedure codes 426 and the candidate code pairs from the procedure-revenue profile matrix. The code combination validation engine 414 may process the candidate code pairs through the domain-specific rules engine 416 and the regulatory compliance engine 418, and may output validated code combinations 424. The code combination validation engine 414 may further receive rules 320A from the rate card and plans data structure 300.

The domain-specific rules engine 416 may execute operations to apply structural rules to the candidate code pairs. The domain-specific rules engine 416 may include a procedure-location validity checker 416A and an inter-procedure dependency constraint checker 416B. The procedure-location validity checker 416A may execute operations to verify whether a given procedure code is permissible for a treatment location identified in the target transactional record. The procedure-location validity checker 416A may function as a location-constraint checker that applies structural rules defining valid relationships between procedures and treatment locations. For example, the procedure-location validity checker 416A may verify that a gastrointestinal (GI) procedure can be assigned to a GI treatment room rather than an incompatible treatment location. The inter-procedure dependency constraint checker 416B may execute operations to verify that the revenue codes selected for a first procedure code on the target transactional record may not conflict with the revenue codes selected for a second procedure code on the same target transactional record. The inter-procedure dependency constraint checker 416B may function as a dependency-constraint checker that applies structural rules to detect inter-procedure revenue code conflicts in a single claim. When the target transactional record includes multiple procedure codes, the inter-procedure dependency constraint checker 416B may evaluate the revenue code assignments across all procedure codes on the target transactional record to detect and eliminate conflicting assignments.

The regulatory compliance engine 418 may execute operations to apply regulatory rules to the candidate code pairs. The regulatory compliance engine 418 may include a Centers for Medicare and Medicaid (e.g., CMS rules submodule 418A, an American Medical Association (AMA) rules submodule 418B, a state rules submodule 418C, and a payer rules submodule 418D). The CMS rules submodule 418A may execute operations to validate candidate code pairs against federal Medicare and Medicaid coding and billing regulations published by CMS. The AMA rules submodule 418B may execute operations to validate candidate code pairs against AMA coding guidelines, including current procedural terminology (CPT) coding rules. The state rules submodule 418C may execute operations to validate candidate code pairs against state-specific Medicaid rules and state regulatory requirements. The payer rules submodule 418D may execute operations to validate candidate code pairs against payer-specific coding and billing rules maintained in the payer policies data store 428D. The regulatory compliance engine 418 may cross-reference the candidate code pairs against each of the CMS rules submodule 418A, the AMA rules submodule 418B, the state rules submodule 418C, and the payer rules submodule 418D, and may flag or eliminate candidate code pairs that violate any applicable regulatory rule.

The NLP-based coding standards update module 420 may execute operations to monitor external data sources for updates to coding standards and regulatory rules. In an embodiment, the NLP-based coding standards update module 420 may execute a natural language processing (NLP) pipeline comprising named entity recognition (NER) extraction, rule mapping, and change detection. The NER extraction component of the NLP-based coding standards update module 420 may execute operations to parse unstructured text of updated coding manuals and extract procedure code identifiers, revenue code references, effective dates, and rule modifications from semi-structured documents published by CMS, the AMA, and state agencies. The rule mapping component of the NLP-based coding standards update module 420 may execute operations to map extracted entities to existing rules in the rules database 428A to identify which validation rules may be affected by the update. The change detection component of the NLP-based coding standards update module 420 may execute operations to compare incoming documents against previously processed versions to identify modified sections. The NLP-based coding standards update module 420 may receive data from the rated and validated coding standards APIs 428C, which may establish API connections with authoritative sources of medical coding standards. The NLP-based coding standards update module 420 may dynamically map extracted parameters to the rules database 428A without disrupting ongoing claims processing by the code combination validation engine 414.

The manual review queue and flagging engine 422 may execute operations to classify each validated candidate code pair into one of three categories, for example, auto-approved, auto-rejected, or flagged-for-manual-review. The classification performed by the manual review queue and flagging engine 422 may be based on a confidence score derived from the weight score and the usage metric stored in the procedure-revenue profile matrix. Candidate code pairs that exceed an upper confidence threshold may be auto-approved by the manual review queue and flagging engine 422. Candidate code pairs that fall below a lower confidence threshold or violate a structural or regulatory rule may be auto-rejected by the manual review queue and flagging engine 422. Candidate code pairs that fall between the upper confidence threshold and the lower confidence threshold may be flagged for manual review by the manual review queue and flagging engine 422, and may be placed in a review queue where a domain analyst may accept, reject, or modify the flagged candidate code pair. The relevance feedback provided by the domain analyst through the manual review queue and flagging engine 422 may be included in the reward signal used by the reinforcement learning engine as ground-truth labels, thereby updating the weight score in the procedure-revenue profile matrix. The output of the code combination validation engine 414 may be the validated code combinations 424, which may represent candidate code pairs that have passed both the structural rule validation by the domain-specific rules engine 416 and the regulatory rule validation by the regulatory compliance engine 418.

The data sources block 428 may provide persistent storage and reference data to both the iPRC 402 and the code combination validation engine 414. The rules database 428A may store the structural rules and the regulatory rules used by the code combination validation engine 414 for validating candidate code pairs. The rate card database 428B may store the rate records, contract terms, effective dates, and exception rules used by the iPRC 402 for computing the expected reimbursement per combination 412. The rated and validated coding standards APIs 428C may provide API connections to external authoritative sources of medical coding standards, and may supply updated coding rules and guidelines to the NLP-based coding standards update module 420. The payer policies data store 428D may store payer-specific coding rules, billing rules, and reimbursement policies used by the payer rules submodule 418D of the regulatory compliance engine 418. The audit trail database 428E may be configured as a tamper-resistant audit data store that stores an audit record for each validated candidate code pair, where each audit record may include an original code assignment of the target transactional record, a recommended code pair corresponding to the validated candidate code pair, a confidence score, a model version identifier, and a timestamp. The audit trail database 428E may provide a persistent record of all code combination recommendations, analyst decisions, and system actions for regulatory compliance and traceability purposes.

The validated code combinations 424 and the expected reimbursement per combination 412 may be transmitted to the combinatorial claims optimizer engine 430. The combinatorial claims optimizer engine 430 may receive the validated and rated candidate code pairs and execute integer programming to identify the combination of procedure codes and revenue codes that maximizes predicted outcome value while satisfying the structural and regulatory rules applied by the code combination validation engine 414.

In operation, the medical code combination optimization system 400 may receive a new medical claim with procedure codes 426. The code combination validation engine 414 may retrieve candidate code pairs from the procedure-revenue profile matrix, where the candidate code pairs may have been generated by processing historical transactional records extracted via the ETL pipeline. The domain-specific rules engine 416 may apply structural rules to the candidate code pairs by first directing the candidate code pairs through the procedure-location validity checker 416A, which may verify that each procedure code may be permissible for the treatment location identified in the target transactional record, and then directing the candidate code pairs through the inter-procedure dependency constraint checker 416B, which may verify that revenue code assignments across multiple procedure codes on the same target transactional record may not conflict with each other. The regulatory compliance engine 418 may then apply regulatory rules by cross-referencing the candidate code pairs against the CMS rules submodule 418A, the AMA rules submodule 418B, the state rules submodule 418C, and the payer rules submodule 418D. Candidate code pairs that violate any structural rule or regulatory rule may be filtered and eliminated by the code combination validation engine 414. The remaining candidate code pairs may be classified by the manual review queue and flagging engine 422 into auto-approved, auto-rejected, or flagged-for-manual-review categories based on the confidence score. The validated code combinations 424 may then be transmitted to the iPRC 402. The rate card resolution engine 404 may select the applicable rate record by matching the effective date and plan through the contract matching by effective date and plan submodule 404A and resolving multi-plan conflicts through the multi-plan conflict resolver submodule 404B. The payment methodology selector 406 may select the applicable rate formula from among the per case submodule 406A, the per diem submodule 406B, the DRG-based percentage submodule 406C, the charges submodule 406D, the per unit submodule 406E, or the fee-schedule formula, based on the payer-plan-location combination. The hierarchical calculation engine 408 may apply the selected rate formula via the priority ranking submodule 408A and aggregate the results via the payment aggregation submodule 408B. The rate card exception engine 410 may evaluate exception rules and may override the standard rate calculation when an exception applies. The expected reimbursement per combination 412 may be produced for each validated candidate code pair. The validated code combinations 424 and the expected reimbursement per combination 412 may then be transmitted to the combinatorial claims optimizer engine 430 for selection of the combination that may maximize the predicted outcome value. An audit record for each validated candidate code pair may be stored in the audit trail database 428E. In another embodiment, the NLP-based coding standards update module 420 may periodically monitor the rated and validated coding standards APIs 428C for updates, may execute the NER extraction, rule mapping, and change detection pipeline, and may dynamically update the rules database 428A without interrupting the validation processing performed by the code combination validation engine 414. In embodiments where the external coding standards 134 publish updates in structured or semi-structured formats, the reinforcement-learning-driven profile matrix optimization system 110 may extract modified rule parameters using deterministic field-extraction logic without invoking trained language models.

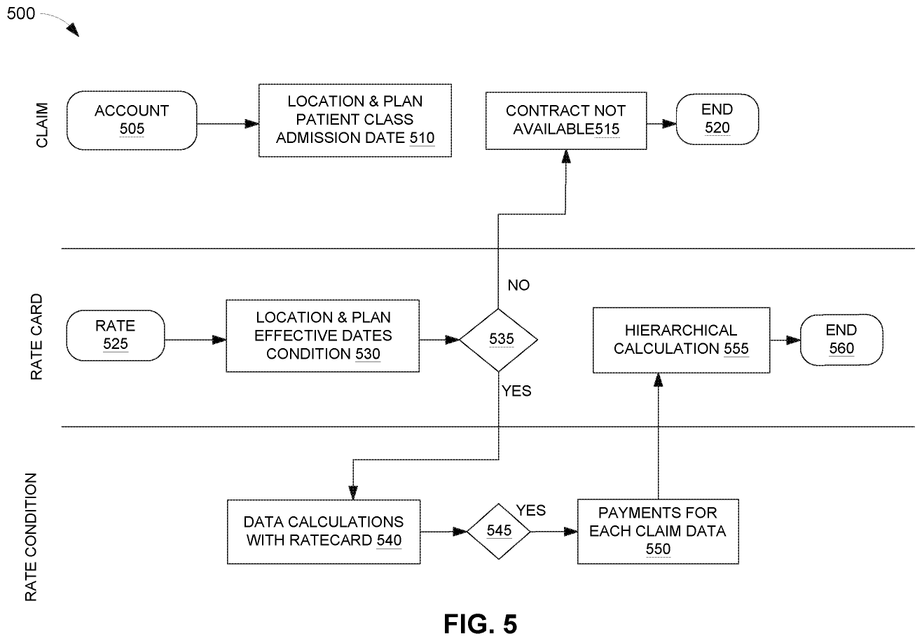

FIG. 5 illustrates a flow diagram showing a data flow process 500 for medical claim processing, according to an exemplary embodiment. FIG. 5 is described in conjunction with FIGS. 1-4. FIG. 5 illustrates a data flow process 500 categorized into, for example, a claim category, a rate card category, and a rate condition category. The data flow process 500 may include an account component 505, a location and plan patient class admission date component 510, a contract not available component 515, a first end state 520, a rate component 525, a location and plan effective dates condition component 530, a first decision point 535, a data calculations with ratecard component 540, a second decision point 545, a payments for each claim data component 550, a hierarchical calculation component 555, and a second end state 560.

In an embodiment, the account component 505 may execute operations to receive or retrieve account-level information associated with a medical claim being processed. The account component 505 may facilitate as the entry point for the data flow process 500, from which claim-specific data attributes may be extracted and forwarded for further processing. The data flow process 500 may then proceed from the account component 505 to the location and plan patient class admission date component 510. The location and plan patient class admission date component 510 may execute operations to gather and organize claim-level details, including the service location, the insurance plan identifier, the patient class designation (indicating whether the encounter may be classified as inpatient, outpatient, or emergency), and the patient's admission date. These attributes extracted by the location and plan patient class admission date component 510 may be used as matching parameters against rate card records in the rate card category. The location and plan patient class admission date component 510 may output the extracted claim attributes downward into the rate card category for contract matching operations.

The rate component 525 may execute operations to access the applicable rate information from a rate data store. The rate component 525 may retrieve rate card records that correspond to the payer and plan associated with the claim being processed. From the rate component 525, the data flow process 500 may proceed to the location and plan effective dates condition component 530. The location and plan effective dates condition component 530 may execute operations to evaluate whether the rate card records retrieved by the rate component 525 have effective date ranges that include the service date or admission date extracted by the location and plan patient class admission date component 510. The location and plan effective dates condition component 530 may further verify that the location and plan attributes from the claim match the location and plan attributes specified in the rate card record. The output of the location and plan patient class admission date component 510 and the output of the location and plan effective dates condition component 530 may converge at the first decision point 535.

The first decision point, 535, may evaluate whether a valid contract match has been identified between the claim attributes and the rate card records. For instance, when the first decision point 535 yields a “NO” outcome, it may indicate that no valid contract match may be found for the combination of location, plan, effective dates, and patient class. The data flow process 500 may route to the contract not available component 515 in the claim category. The contract not available component 515 may indicate that the rate card and plans data structure 300 may lack sufficient contract information to process the claim, and the data flow process 500 may terminate at the first end state 520. The first end state 520 may represent a terminal condition in which the claim may not be processed further through the rate calculation pipeline due to the absence of a matching contract record.

When the first decision point 535 yields a “YES” outcome, it may indicate that a valid contract match has been identified, and the data flow process 500 may proceed to the rate condition category. In the rate condition category, the data calculations with the ratecard component 540 may execute operations to perform reimbursement computations using the matched rate card information. The data calculations with the ratecard component 540 may apply the applicable payment methodology, for example, per case, per diem, diagnosis-related-group-based, percentage of charges, per unit, or fee schedule calculations, based on the payment type specified in the matched rate card record. The data calculations with the ratecard component 540 may also apply rate exception rules where the matched contract record specifies exceptions spanning particular plans, service locations, or contract terms. The output of the data calculations with the ratecard component 540 may be evaluated at the second decision point 545.

When the second decision point 545 yields a “YES” outcome, it may indicate that the calculation criteria have been satisfied, and the data flow process 500 may proceed to the payments for each claim data component 550. The payments for each claim data component 550 may be executed operations that determine and store the computed payment amounts for each line item or charge element in the claim, based on the rate calculations performed by the data calculations with the ratecard component 540. The payments for each claim data component 550 may feed upward into the hierarchical calculation component 555 in the rate card category. The hierarchical calculation component 555 may execute operations that apply a priority-based structure to the payment calculations. The plan-specific rate records may be prioritized over location-specific rate records, and location-specific rate records may be prioritized over general rate records. The hierarchical calculation component 555 may aggregate the payment amounts computed by the payments for each claim data component 550 across all line items and apply the hierarchical ordering to resolve any conflicts among overlapping rate records. Upon completion of the hierarchical calculation component 555, the data flow process 500 may terminate at the second end state 560, at which point the predicted outcome value for the processed claim may be finalized.

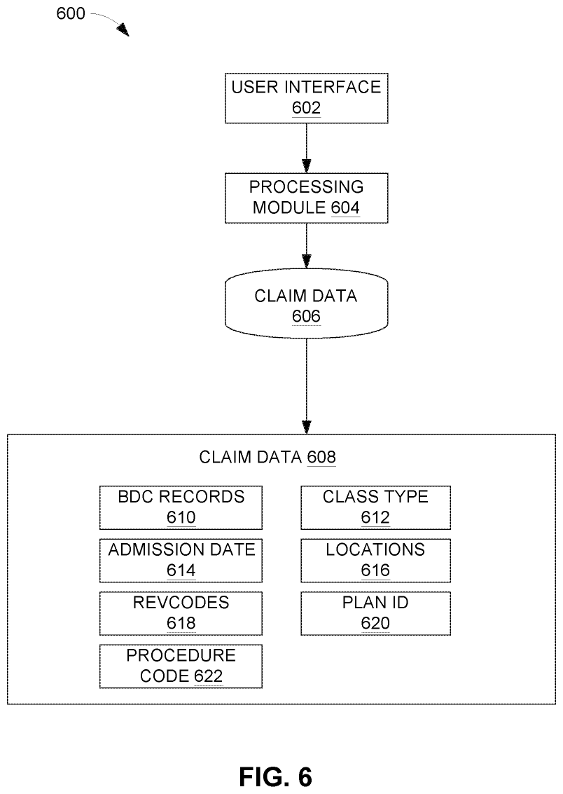

FIG. 6 illustrates a block diagram 600 showing a consolidated system for rates, claims, and query preparation execution, according to an exemplary embodiment. FIG. 6 is described in conjunction with FIG. 1-5. In an embodiment, the block diagram 600 illustrates a consolidated system that may include a user interface 602, a processing module 604, a claim data input 606, a structured claim data block 608 including BDC records 610, class type 612, admission date 614, locations 616, RevCodes 618, plan ID 620, and procedure code 622, together with a rate card and plans data section, a reference data section, a multi conditions and joins section, a final table, and a ranking section comprising a consolidated table, a hierarchical ranking output, an analytics top rank module, and a visualization component.

The claim data input 606 is received through the user interface 602 and routed through the processing module 604, which structures the incoming claim data into the claim data block 608. The claim data block 608 organizes the claim attributes into BDC records 610, class type 612, admission date 614, locations 616, RevCodes 618, plan ID 620, and procedure code 622. A reference data section associated with the consolidated system 600 provides group codes, FS codes, and exhibits, which are resolved into a group scheme, a fee schedule, and an exhibits table. These reference data outputs are made available to the multi conditions and joins section for use during query preparation.

The consolidated system is communicatively coupled with the claim data block 608 and the reference data section to the rate card and plans data section, such that rate records, including rate data for different locations, RevCodes, procedure codes, FS codes, DRG codes, and other codes, are accessible during multi-condition processing. The claim data attributes and resolved reference data are matched against rate compact and other data, including exhibits, rate records, and fee schedule tables, in the multi conditions and joins section. The joins operation is performed on plan ID, patient class, location, and admission date as primary join keys, following which unique formulas may be identified and added to a final query. Query execution may produce either a processed output directed to a final table or an error condition that terminates the query. The final table output is passed to the ranking section via a hierarchical ranking pathway, where a consolidated table is ranked, and the top-ranked results are surfaced through the analytics top rank module and presented through the visualization component.

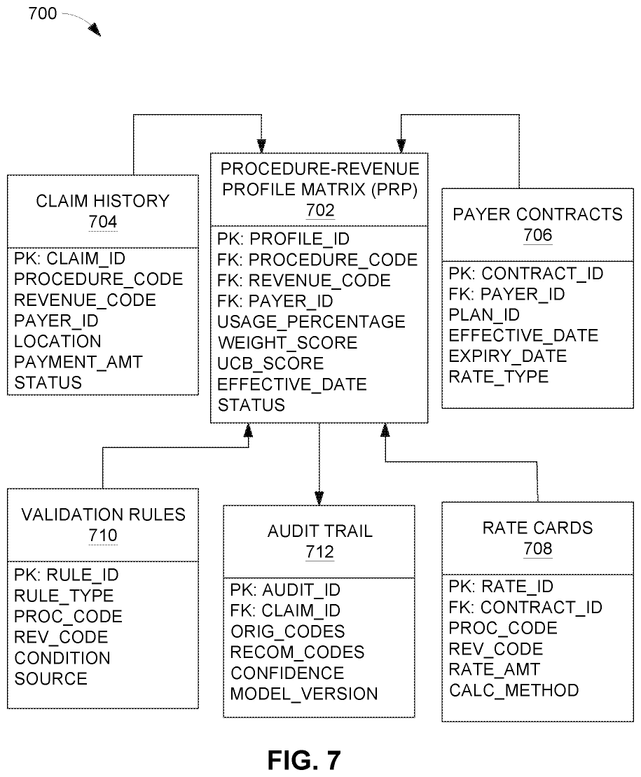

FIG. 7 illustrates a database schema 700 for the reinforcement-learning-driven profile matrix optimization system, according to an exemplary embodiment. FIG. 7 is described in conjunction with FIGS. 1-6. FIG. 7 illustrates a database schema 700 showing the structure and interrelationships of data entities, for example, a PRP matrix 702, a claim history 704, payer contracts 706, rate cards 708, validation rules 710, and an audit trail 712.

The PRP matrix 702 may be a data structure that includes a relational table with a primary key field designated PROFILE_ID, foreign key fields referencing PROCEDURE_CODE, REVENUE_CODE, and PAYER_ID, and additional attribute fields including USAGE_PERCENTAGE, WEIGHT_SCORE, UCB_SCORE, EFFECTIVE_DATE, and STATUS. The WEIGHT_SCORE and UCB_SCORE fields may store values computed by the reinforcement learning module to support exploration-exploitation balancing during code combination selection. The USAGE_PERCENTAGE field may record the historical frequency with which a given procedure code and revenue code pairing has been submitted and approved, and the STATUS field may indicate whether a given profile record may be active or inactive at the time of query execution. The PRP matrix 702 may be partitioned by the PAYER_ID foreign key, so that a given procedure code may have a different WEIGHT_SCORE for a first payer than for a second payer.

The claim history 704 may be a data structure represented as a relational table having a primary key field designated CLAIM_ID, and attribute fields including PROCEDURE_CODE, REVENUE_CODE, PAYER_ID, LOCATION, PAYMENT_AMT, and STATUS. The claim history 704 may facilitate identifying the source from which historical transactional records can be extracted, transformed, and loaded into the data processing system to train the machine learning engines 112 and construct the PRP matrix 702.

The payer contracts 706 may be a data structure represented as a relational table having a primary key field designated CONTRACT_ID, a foreign key field referencing PAYER_ID, and attribute fields including PLAN_ID, EFFECTIVE_DATE, EXPIRY_DATE, and RATE_TYPE. The payer contracts 706 may store the contractual terms governing reimbursement arrangements between a healthcare facility and a given payer, and the RATE_TYPE field may identify the payment methodology applicable to claims under a given contract.

The rate cards 708 may be a data structure represented as a relational table having a primary key field designated RATE_ID, a foreign key field referencing CONTRACT_ID, and attribute fields including PROC_CODE, REV_CODE, RATE_AMT, and CALC_METHOD. The rate cards 708 may store procedure-specific and revenue-code-specific rate amounts, with the calculation method used to determine expected reimbursement, and may be associated with a given payer contract via the CONTRACT_ID foreign key.

The validation rules 710 may be structured as a relational table having a primary key field designated RULE_ID, and attribute fields including RULE_TYPE, PROC_CODE, REV_CODE, CONDITION, and SOURCE. The validation rules 710 may store structural rules and regulatory rules against which candidate code pairs from the PRP matrix 702 may be validated prior to reimbursement calculation. The SOURCE field may record the origin of each rule, including external coding standards bodies such as the CMS or the AMA.

The audit trail 712 may be structured as a relational table having a primary key field designated AUDIT_ID, a foreign key field referencing CLAIM_ID from the claim history 704, and attribute fields including ORIG_CODES, RECOM_CODES, CONFIDENCE, and MODEL_VERSION. The audit trail 712 may store a tamper-resistant record for each code-combination recommendation, capturing the original code assignment, the recommended code pair, a confidence score, and the model version identifier of the model that generated the recommendation.

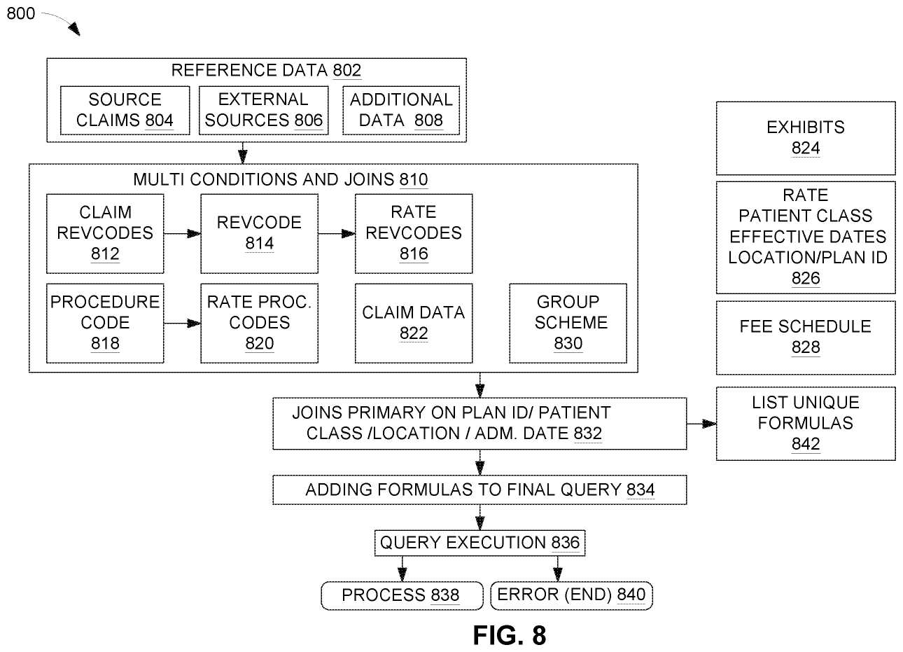

FIG. 8 illustrates a block diagram 800 showing a query preparation and execution system for medical claims processing, according to an exemplary embodiment. FIG. 8 is described in conjunction with FIGS. 1-7. FIG. 8 illustrates a block diagram 800 showing a query preparation and execution for processing medical claims. In an embodiment, FIG. 8 shows a reference data component 802 comprising source claim 804, external sources 806, and additional data 808, a multi conditions and joins module 810 including claim revenue codes 812, revenue code 814, rate revenue codes 816, procedure code 818, rate procedure codes 820, claim data 822, exhibits 824, rate patient class effective dates location and plan identifier 826, fee schedule 828, and group scheme 830, a joins block 832 operating on primary keys comprising plan identifier, patient class, location, and admission date, a list unique formulas block 842, an adding formulas to final query block 834, a query execution block 836, a process block 838, and an error (end) block 840.

The reference data component 802 may execute operations to aggregate data from, for example, source claim 804, external sources 806, and additional data 808, and send a unified data feed to the multi-conditions and joins module 810. In the multi-condition and joins module 810, claim revenue codes 812 are mapped to revenue code 814 on the claim side and to rate revenue codes 816 on the rate contract side. Procedure code 818 is similarly matched against rate procedure codes 820 to establish correspondence between submitted claim procedure identifiers and rate-side procedure identifiers. Claim data 822, which may include patient class, admission date, location, and plan identifier attributes, is presented as a structured data input in the module multi conditions and joins module 810. Group scheme 830 provides group scheme conditions, and fee schedule 828 provides fee schedule values against which claim data 822 may be evaluated. Exhibits 824 are included as supplemental contractual reference data, and rate patient class effective dates, location, and plan identifier 826 provide temporal and geographic rate-binding attributes from the rate contract side.

The list unique formulas 842 component identifies and enumerates distinct formula expressions applicable to the processed record set and supplies these formula references to the joins block 832. The joins 832 component consolidates the multi-condition outputs by executing a primary join on plan identifier, patient class, location, and admission date, thereby producing a joined dataset that reflects the intersection of claim-side and rate-side data. The adding formulas to the final query 834 component receives the joined dataset and appends the formula expressions identified in the list unique formulas 842 to form a complete executable query. The query execution 836 executes the assembled query and routes the outcome along two paths. When execution succeeds, the process block 838 receives the output for downstream claims processing. When execution encounters an exception, the error (end) block 840 terminates the processing sequence.

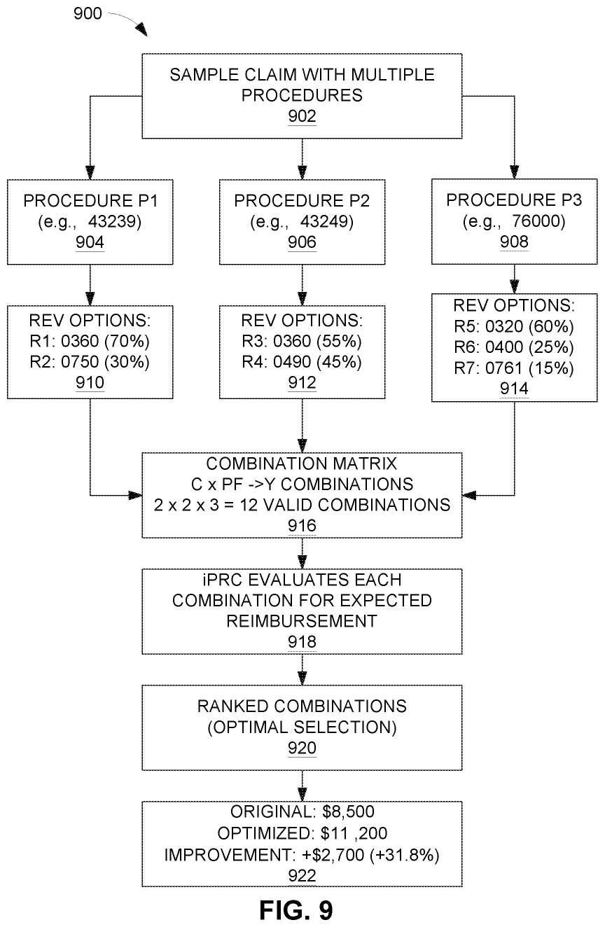

FIG. 9 illustrates a block diagram 900 showing a worked example of the combinatorial claims optimization mechanism, according to an exemplary embodiment. FIG. 9 is described in conjunction with FIGS. 1-8. FIG. 9 illustrates a block diagram 900 showing a combinatorial claims optimization mechanism. In an embodiment, FIG. 9 shows the following components, including a sample claim with multiple procedures 902, procedure P1 904, procedure P2 906, procedure P3 908, a revenue options block for P1 910, a revenue options block for P2 912, a revenue options block for P3 914, a combination matrix 916, an iPRC evaluation block 918, a ranked combinations block 920, and a reimbursement comparison result block 922.

The sample claim with multiple procedures 902 represents a healthcare claim including three concurrently billed procedures. Procedure P1 904, identified by an example procedure code 43239, is associated with revenue options block 910, which lists two candidate revenue codes, R1 0360 with a historical usage weighting of 70% and R2 0750 with a historical usage weighting of 30%. Procedure P2 906, identified by example procedure code 43249, is associated with revenue options block 912, which lists R3 0360 at 55% and R4 0490 at 45%. Procedure P3 908, identified by example procedure code 76000, is associated with revenue options block 914, which lists R5 0320 at 60%, R6 0400 at 25%, and R7 0761 at 15%. The revenue options blocks 910, 912, and 914 reflect profiled procedure-revenue pairings derived from historical claim data. Each percentage represents the frequency with which a given revenue code has been used in approved claims for the corresponding procedure code.

The combination matrix 916 receives the revenue option sets from all three procedure branches and constructs the complete set of valid procedure-revenue combinations by applying the formula

C*PF→Y (6)
where C represents the claim, PF represents the profiled revenue code options per procedure, and Y represents the resulting valid combinations. For example, for the three procedures with two, two, and three revenue code options, the combination matrix 916 computes 2×2×3=12 valid combinations. Each combination represents a distinct assignment of one revenue code to each procedure on the claim.

The iPRC evaluation 918 receives, for example, the 12 valid combinations generated by the combination matrix 916 and evaluates each combination for its expected transaction outcome value. The intelligent payment rate calculator applies the applicable payment methodology, including per case, per diem, percentage of charges, per unit aggregate, and fee schedule methods, based on payer-plan-location parameters associated with the claim, and computes an expected payment amount for each combination.

The ranked combinations 920 receive the expected transaction outcome values computed by the iPRC evaluation block 918, and all 12 combinations are ranked in descending order of expected reimbursement, with the highest-valued combination identified as the optimal selection. The reimbursement comparison result 922 represents the output of the optimization mechanism, comparing the original submitted reimbursement of $8,500 with the optimized reimbursement of $11,200, an improvement of $2,700 (31.8%).

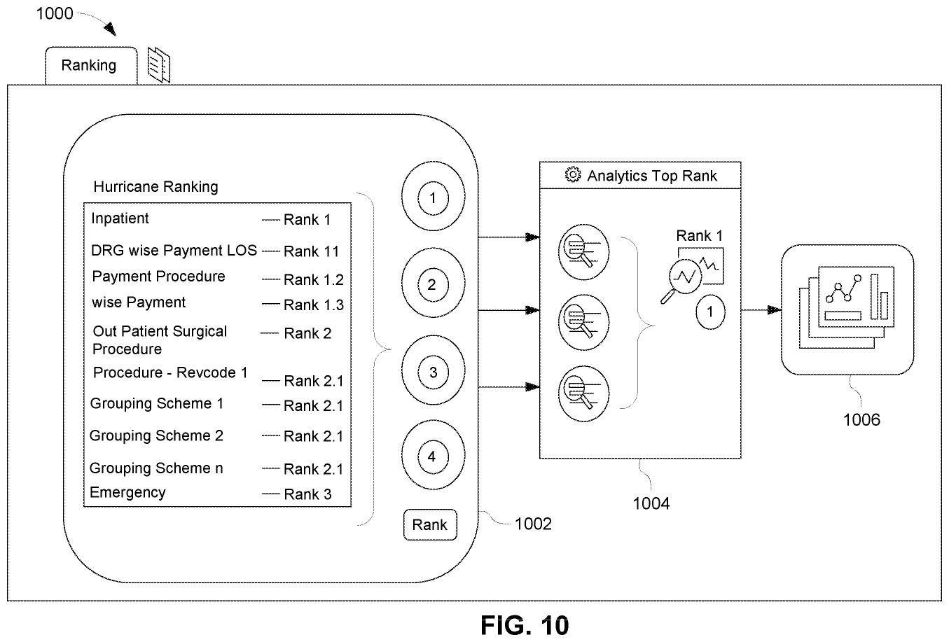

FIG. 10 illustrates a user interface 1000 showing a ranking mechanism for prioritizing and processing medical claim code combinations, according to an exemplary embodiment. FIG. 10 is described in conjunction with FIGS. 1-9. FIG. 10 illustrates a user interface 1000 showing a ranking mechanism for prioritizing and processing medical code combinations. In an embodiment, FIG. 10 shows components or elements including a consolidated table 1002 including a hurricane ranking structure, an analytics top rank module 1004, and a visualization output 1006.

The consolidated table 1002 may process and display claim type entries in a hierarchical ranking structure referred to as “Hurricane Ranking.” In the consolidated table 1002, the ranked entries are assigned rank identifiers that reflect their relative priority level for reimbursement impact. Inpatient claims are assigned Rank 1, representing the highest priority tier. In Rank 1, sub-ranks are defined as, for example, DRG-wise payment is assigned Rank 1.1, LOS payment is assigned Rank 1.2, and procedure-wise payment is assigned Rank 1.3. The outpatient's surgical procedure is assigned Rank 2. Procedure-revenue code 1 is assigned Rank 2.1, and grouping scheme 1, grouping scheme 2, and grouping scheme n are each assigned Rank 2.1, reflecting equivalent weighting in that tier. Emergency claims are assigned Rank 3. Rank positions with higher values in the hierarchy, particularly Rank 1 entries, may be treated as higher-impact combinations for reimbursement optimization purposes.

From the consolidated table 1002, ranked entries are passed to four rank selector positions labeled 1, 2, 3, and 4 in the “Ranks” column. Rank selectors 1, 2, and 3 feed into the analytics top rank 1004. The analytics top rank module 1004 receives the top-ranked entries and processes them through three analytics processing elements represented as data query icons. The output of the analytics top rank 1004 identifies Rank 1 as the top-priority result, indicated by a search-and-analytics icon labeled “1.” The analytics top rank module 1004 then passes its output to the visualization output 1006.

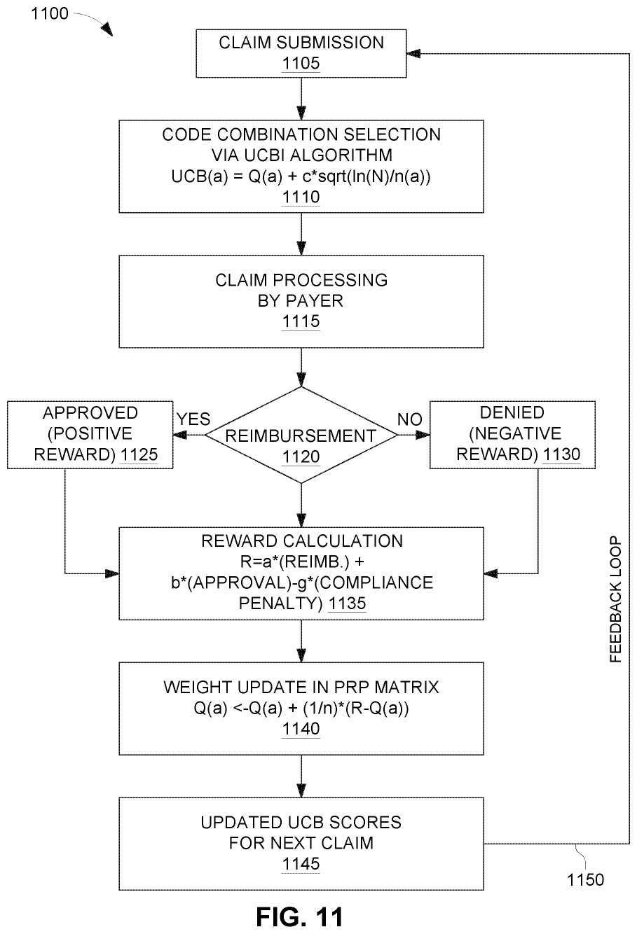

FIG. 11 illustrates a flow diagram showing a process 1100 for reinforcement learning feedback loop, according to an exemplary embodiment. FIG. 11 illustrates a flow diagram of a process 1100 for reinforcement learning feedback loop. In an embodiment, FIG. 11 shows a claim submission stage 1105, a code combination selection stage 1110, a claim processing stage 1115, a reimbursement decision point 1120, an approved outcome branch 1125, a denied outcome branch 1130, a reward calculation stage 1135, a weight update stage 1140, an updated UCB scores stage 1145, and a feedback loop path 1150 connecting the updated UCB scores stage 1145 back to the claim submission stage 1105. The code combination selection stage 1110 may apply the contextual bandit algorithm to the weight scores stored in the PRP matrix 206 of FIG. 2A. The UCB algorithm at the code combination selection stage 1110 may be expressed defined by equation (3), where ‘Q(a)’ represents the estimated cumulative reward for a candidate code pair a, ‘N represents the total count of selections across all validated candidate code pairs, ‘n(a)’ represents the count of times the candidate code pair a has been selected, and c represents an exploration parameter governing the balance between an exploration score and an exploitation score. The exploration-exploitation score computed by the code combination selection stage 1110 may balance or scale the estimated cumulative reward for each validated candidate code pair against a degree of uncertainty derived from the ratio of the total count of selections across all validated candidate code pairs to the count of times the validated candidate code pair has been selected, as recited in the claims of the present disclosure.

At the claim processing stage 1115, a submitted claim may be processed by a payer, and the reimbursement decision point 1120 may bifurcate the outcome into the approved outcome 1125 or the denied outcome 1130. The approved outcome 1125 may generate a positive reward signal, and the denied outcome 1130 may generate a negative reward signal. Both may converge at the reward calculation stage 1135, where the composite reward R may be computed using equation (1). The coefficient α may weight a reimbursement component, the coefficient β may weight an approval-indicator component, and the coefficient γ may weight a constraint-violation penalty component. The weighting coefficients may be initialized from hierarchical contract rules stored in the rate card database 214D of FIG. 2B and may be configurable to reflect payer-specific or location-specific reimbursement behavior.

At the weight update stage 1140, the weight score for the selected candidate code pair in the PRP matrix 206 may be updated using the incremental averaging rule as defined in equation (2), where ‘n’ represents the count of times the candidate code pair a has been selected and ‘R’ represents the composite reward computed at the reward calculation stage 1135. The reinforcement learning engine may incrementally adjust the weight score toward the reward signal in proportion to the inverse of the count of times the validated candidate code pair has been selected, producing an adjusted weight score. The ‘1/n’ learning rate may cause early observations to carry proportionally greater influence when data for a given candidate code pair is sparse, and the update rule may converge toward the true expected reward as n increases without requiring full retraining of the machine learning engines 112. At the updated UCB scores stage 1145, the revised Q(a) value may be used to recompute the UCB score for the affected candidate code pair, and the recomputed score may be propagated through the feedback loop path 1150 back to the claim submission stage 1105 for use in selecting the code combination for a subsequent transactional record. Through successive iterations of the reinforcement learning feedback loop 1100, the weight scores in the PRP matrix 206 may be continuously refined based on downstream outcome data, including the approval status and the transaction outcome associated with processed transactional records. The candidate code pairs with higher historical approval rates and reimbursement yields may receive progressively higher adjusted weight scores and may be more frequently selected by the code combination selection stage 1110.

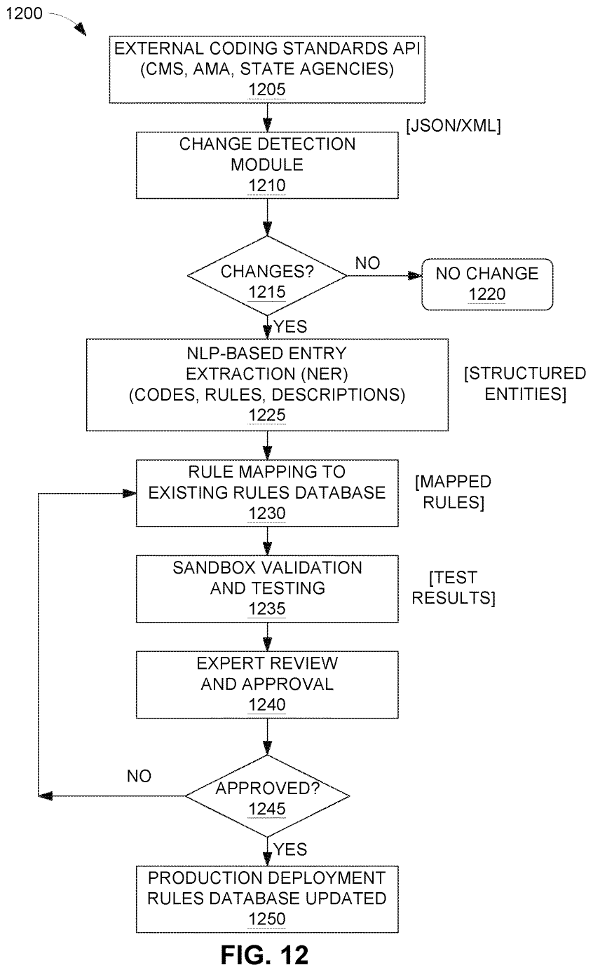

FIG. 12 illustrates a mechanism 1200 for updating coding standards, according to an exemplary embodiment. FIG. 12 is described in conjunction with FIGS. 1-11. The coding standards update mechanism 1200 shows an external coding standards API 1205, a change detection module 1210, a changes decision point 1215, a no change state 1220, an NLP-based entry extraction module 1225, a rule mapping module 1230, a sandbox validation and testing module 1235, an expert review and approval module 1240, an approved decision point 1245, and a production deployment state 1250.

The external coding standards API 1205 maintains programmatic connections to authoritative sources of medical coding standards, including the CMS, the AMA, and state agencies. Data received through the external coding standards API 1205 may be formatted in JSON or XML, as indicated by the [JSON/XML] annotation in FIG. 12. The external coding standards API 1205 is the upstream data source from which regulatory changes are ingested into the coding standards update system.

The change detection module 1210 receives data transmitted by the external coding standards API 1205 and compares it against the current state of the rules database 428A. Based on the comparison, a determination is made as to whether any modifications have occurred to coding standards, procedure code identifiers, revenue code references, effective dates, or rule parameters. Text similarity scoring may be applied by the change detection module 1210 to detect modified sections in incoming coding manual updates relative to prior versions stored in the rules database 428A. At the changes decision point 1215, the coding standards update mechanism 1200 evaluates whether substantive changes have been detected. Where no changes are detected, the no change state 1220 is reached, and no further processing is performed for that update cycle. When changes are detected, the mechanism 1200 proceeds to the NLP-based entry-extraction module 1225.

In an embodiment, the NLP-based entry extraction module 1225 applies named entity recognition (NER) and text classification to parse the incoming update data and extract structured entities from structured and semi-structured update feeds. The structured entities extracted by the NLP-based entry extraction module 1225 may include updated procedure codes, revenue codes, modified rule descriptions, and effective dates, as shown (CODES, RULES, DESCRIPTIONS) in FIG. 12 and the [STRUCTURED ENTITIES]. Text classification performed by the NLP-based entry extraction module 1225 categorizes each extracted change as a new rule, a modification to an existing rule, or a deprecation of a previously active rule. The NLP-based entry extraction module 1225 corresponds to the NLP-based coding standards update module 420, which executes operations for NER extraction, rule mapping, and change detection.

The rule mapping module 1230 receives the structured entities output by the NLP-based entry extraction module 1225 and maps the received entities against existing entries in the rules database 428A. Based on the mapping, the specific validation rules may identify those affected by the detected changes. The mapped rules output by the rule mapping module 1230 are labeled [MAPPED RULES] and forwarded to the sandbox validation and testing module 1235. The sandbox validation and testing module 1235 applies the mapped rules to a subset of historical claims data in a test environment that mirrors the production configuration of the reinforcement-learning-driven profile matrix optimization system 110. Test results produced by the sandbox validation and testing module 1235 are indicated by the [TEST RESULTS] label and measure the impact of the candidate rule changes against known claim outcomes from the historical test subset.

The expert review and approval module 1240 receives the test results from the sandbox validation and testing module 1235, and a designated coding expert or administrator reviews the proposed rule changes and the associated sandbox test results before any update is propagated to the production environment. At the approved decision point 1245, a determination is made on whether the proposed changes have been approved. Where approval is not granted, the workflow returns to the rule mapping module 1230, allowing the extracted entities to be re-evaluated and remapped before re-submission for sandbox validation and expert review. Where approval is granted, the production deployment state 1250 is reached, and the rules database 428A is updated with the approved rule modifications. The updated rules are made available to the code combination validation engine 414, the regulatory compliance engine 418, and all other components of the reinforcement-learning-driven profile matrix optimization system 110 that depend on the rules database 428A for validation processing. The production deployment state 1250 enables rule changes to be integrated into the production environment without disrupting ongoing claims processing by the combinatorial claims optimizer engine 118, the code combination validation engine 414, and the intelligent payment rate calculator (iPRC) 402.

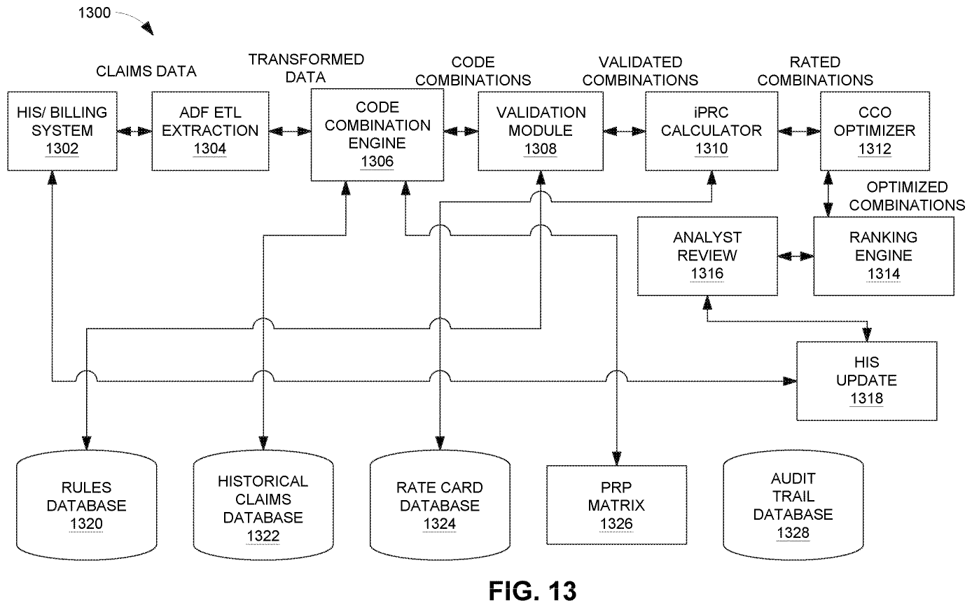

FIG. 13 illustrates an end-to-end claim processing pipeline 1300, according to an exemplary embodiment. FIG. 13 is described in conjunction with FIGS. 1-12. In an embodiment, FIG. 13 illustrates an end-to-end claim processing pipeline 1300, which shows the sequential data flow through the components of the reinforcement-learning-driven profile matrix optimization system 110. The end-to-end claim processing pipeline 1300 may include a hospital information system (HIS)/billing system 1302, an Azure Data Factory (ADF), ETL extraction module 1304, a code combination engine 1306, a validation module 1308, an iPRC calculator 1310, a code combination optimizer (CCO) optimizer 1312, a ranking engine 1314, an analyst review module 1316, and a HIS update module 1318. The end-to-end claim processing pipeline 1300 further references a rules database 1320, a historical claims database 1322, a rate card database 1324, a procedure-revenue profile matrix (PRP) matrix 1326, and an audit trail database 1328.

The HIS/billing system 1302 corresponds to the healthcare data systems (e.g., 102, 104, 106) and may serve as the source of claims data. The ADF ETL extraction module 1304 corresponds to the data extraction and loading module 108 described with reference to FIG. 1 and may receive claims data from the HIS/billing system 1302. The ADF ETL extraction module 1304 may map claims data from hospital coding nomenclatures into a standardized format, and may store the transformed data in one or more columnar storage partitions across multiple distributed databases. The output of the ADF ETL extraction module 1304 is labeled as transformed data and may be passed to the code combination engine 1306.

The code combination engine 1306 corresponds to the machine learning engines 112, which may receive the transformed data and may access the historical claims database 1322 and the PRP matrix 1326 to generate code combinations. The code combinations generated by the code combination engine 1306 may be derived through principal component analysis (PCA) dimensionality reduction, association rule mining, and profile matching and scoring operations. The output of the code combination engine 1306 is labeled as code combinations and may be forwarded to the validation module 1308.

The validation module 1308 corresponds to the code combination validation module 116, which may receive code combinations and access the rules database 1320 to validate each candidate code pair against the structural rules and regulatory rules. The structural rules may include the location-constraint checker and the dependency-constraint checker, and the regulatory rules may include CMS, AMA, state, and payer rules. Candidate code pairs that violate any structural rule or regulatory rule may be filtered and eliminated. Each validated candidate code pair may be classified as auto-approved, auto-rejected, or flagged for manual review. The output of the validation module 1308 is labeled as validated combinations and may be forwarded to the iPRC calculator 1310.

The iPRC calculator 1310 may receive validated combinations and access the rate card database 1324 to determine an expected reimbursement amount for each validated candidate code pair using a hierarchical calculation engine. The rate formula may be selected based on a combination of the payer identifier, a plan identifier, and a location identifier associated with the target transactional record. The output of the iPRC calculator 1310, labeled as rated combinations, may be forwarded to the CCO optimizer 1312.

The CCO optimizer 1312 corresponds to the combinatorial claims optimizer engine 118, which may receive the rated combinations and may apply integer programming for combinatorial optimization to identify the code combination that maximizes the predicted outcome value subject to the structural and regulatory constraints applied by the validation module 1308. The output of the CCO optimizer 1312 is labeled as optimized combinations and may be forwarded to the ranking engine 1314.

The ranking engine 1314 may receive the optimized combinations and may rank the candidate code pairs based on the adjusted weight scores produced by the reinforcement learning engine executing the multi-armed bandit algorithm. The ranking engine 1314 may correspond to the ranked code recommendations 130 and the hurricane ranking methodology described with reference to FIG. 10. The ranked output of the ranking engine 1314 may be forwarded to the analyst review module 1316.

The analyst review module 1316 corresponds to the user interface/analyst review 132 and the revenue cycle analyst 216. The analyst review module 1316 may present each ranked candidate code pair to a domain analyst for acceptance, rejection, or modification. Relevance feedback from the domain analyst may be included as a ground-truth label in the reward signal used by the reinforcement learning engine to update the weight score in the PRP matrix 1326, thereby closing the reinforcement learning feedback loop. Each recommendation, the original code assignment, the recommended code pair, the confidence score, and the model version identifier may be recorded in the audit trail database 1328.

The HIS update module 1318 may receive the final validated and approved candidate code pair and may automatically transmit a structured data output comprising the validated candidate code pair, the predicted outcome value, a confidence score derived from the adjusted weight score, and a model version identifier to the external data (e.g., healthcare related data) system, corresponding to the HIS/billing system 1302. The integration into the target transactional record may be executed via an API coupled to the multiple distributed databases.

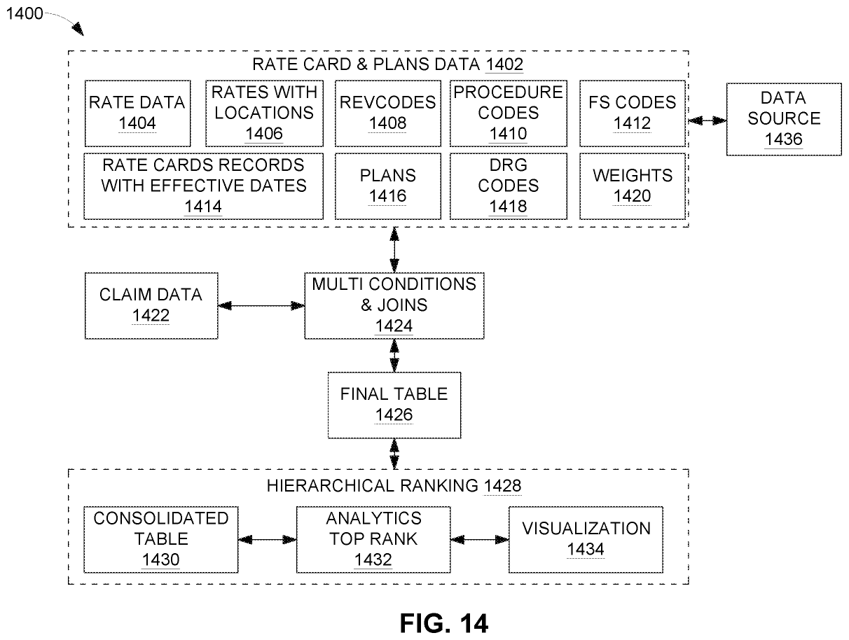

FIG. 14 illustrates a block diagram showing a per-hospital data processing pipeline 1400, according to an exemplary embodiment. FIG. 14 is described in conjunction with FIGS. 1-13. FIG. 14 illustrates a per-hospital data processing pipeline 1400 that shows the end-to-end flow from rate and claim data ingestion through hierarchical ranking and visualization output in a hospital deployment. FIG. 14 shows a rate card and plans data block 1402, a data source 1436, a claim data block 1422, a multi-conditions and joins module 1424, a final table 1426, a hierarchical ranking module 1428, a consolidated table 1430, an analytics top rank module 1432, and a visualization output 1434. The rate card and plans data block 1402 comprises rate data 1404, rates with locations 1406, RevCodes 1408, procedure codes 1410, FS codes 1412, rate cards records with effective dates 1414, plans 1416, DRG codes 1418, and weights 1420.

The rate data 1404 may store base rate values, and the rates with locations 1406 may store location-differentiated rate records. The RevCodes 1408 and the procedure codes 1410 may store the revenue code and procedure code reference tables, respectively. The FS codes 1412 may store fee schedule classification codes. The rate cards records with effective dates 1414 may store temporally bounded rate card entries, such that applicable rate records may be resolved based on the service date of a target transactional record. The plans 1416 may store plan-level parameters associated with different payment methodologies. The DRG codes 1418 may store DRG classification codes, and the weights 1420 may store weights used in reimbursement calculations.

The claim data block 1422 may receive structured claim records from the data source 1436. The data source 1436 may represent the hospital-specific data store from which historical and incoming transactional records may be retrieved. The claim data block 1422 and the rate card and plans data block 1402 may both feed into the multi conditions and joins module 1424. The claim attributes may be matched against rate records using join operations on plan identifier, patient class, location, and admission date, consistent with the multiple conditions and joins processing. The output of the multi conditions and joins module 1424 may be directed to the final table 1426, which may store the resolved claim-rate combinations.

The final table 1426 may feed into the hierarchical ranking module 1428, where the resolved combinations may be ranked according to the hurricane ranking scheme. The ranked output may be organized into the consolidated table 1430, from which the top-ranked entries may be passed to the analytics top rank module 1432. The analytics top rank module 1432 may surface the highest-priority code-rate combinations, and the output of the analytics top rank module 1432 may be forwarded to the visualization output 1434 for presentation as a dashboard report.

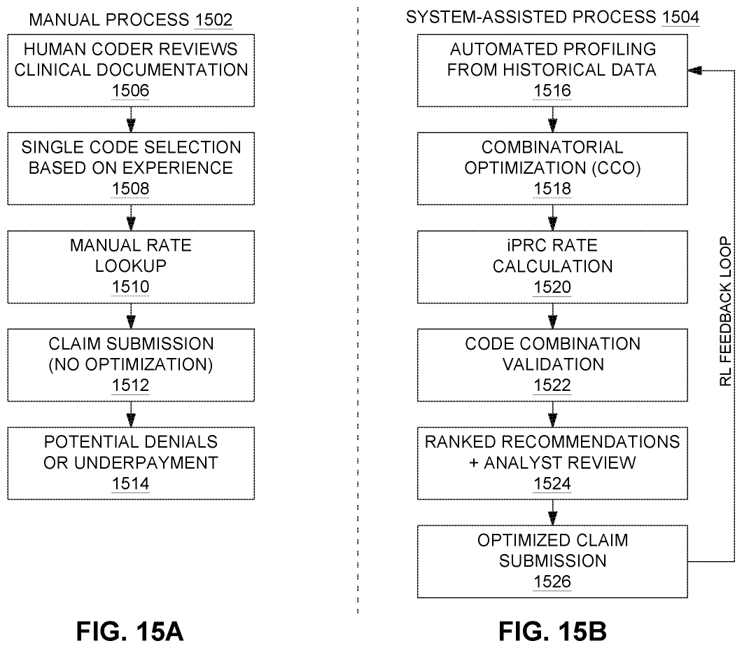

FIG. 15A and FIG. 15B illustrates a side-by-side comparison between a manual process 1502 and a system-assisted process 1504 for medical claim coding and submission, according to an exemplary embodiment. FIGS. 15A and 15B are described in conjunction with FIGS. 1-14. In an embodiment, the manual process 1502 is shown in FIG. 15A may include human coder reviews clinical documentation 1506, single code selection based on experience 1508, manual rate lookup 1510, claim submission with no optimization 1512, and potential denials or underpayment 1514. The system-assisted process 1504 shown in FIG. 15B may include automated profiling from historical data 1516, CCO 1518, iPRC rate calculation 1520, code combination validation 1522, ranked recommendations with analyst review 1524, optimized claim submission 1526, and an RL feedback loop returning from the submission stage back to the CCO 1518.

In the manual process 1502, clinical documentation review 1506 is performed by a human coder, from which a single code combination may be selected based solely on individual experience 1508. A manual rate lookup 1510 may then be performed without reference to payer-specific contract rules or combinatorial alternatives, after which the claim may be submitted without optimization 1512. The absence of automated validation and combinatorial analysis in the manual process 1502 may result in potential denials or underpayment 1514.

In the system-assisted process 1504, historical claims data may be processed by the automated profiling module 1516 to construct a procedure-revenue profile matrix. The CCO engine 1518 may then generate candidate code combinations from the profile matrix. The iPRC rate calculation 1520 module may compute an expected transaction outcome value for each candidate combination. The code combination validation module 1522 may filter candidate pairs against structural and regulatory rules stored in the rules database. Validated and rated combinations may then be presented as ranked recommendations 1524 for analyst review before the optimized claim may be submitted 1526. The RL feedback loop may return a reward signal from the submission outcome to the CCO engine 1518, enabling the procedure-revenue profile matrix weights to be updated incrementally for subsequent claims.

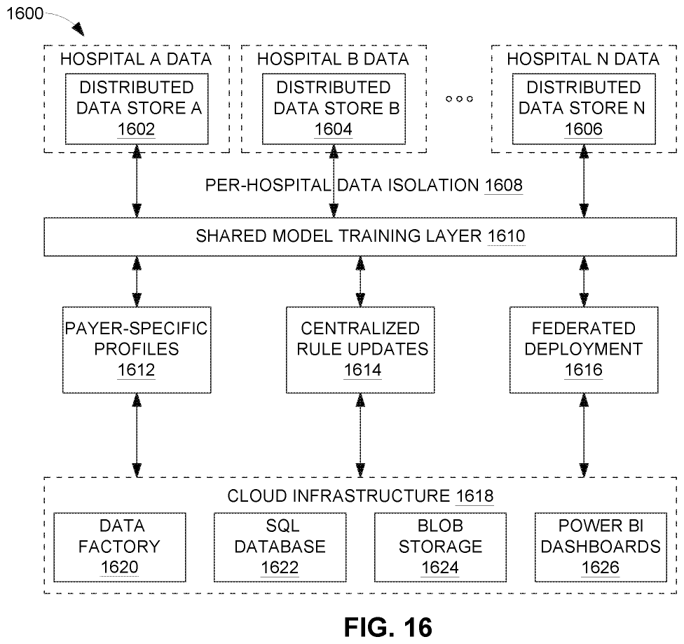

FIG. 16 illustrates a deployment architecture 1600 of the reinforcement-learning-driven profile matrix optimization system 110, according to an exemplary embodiment. FIG. 16 is described in conjunction with FIGS. 1-15. In an embodiment, the deployment architecture 1600 may include a distributed data store A 1602, a distributed data store B 1604, a distributed data store N 1606, a per-hospital data isolation layer 1608, a shared model training layer 1610, payer-specific profiles 1612, centralized rule updates 1614, a federated deployment module 1616, and a cloud infrastructure layer 1618. The cloud infrastructure layer 1618 comprises a data factory 1620, a SQL database 1622, blob storage 1624, and Power BI dashboards 1626.

The distributed data store A 1602 may store hospital A data, the distributed data store B 1604 may store hospital B data, and the distributed data store N 1606 may store hospital N data. Each of the distributed data stores 1602, 1604, and 1606 may correspond to the columnar storage partitions of the distributed databases. Each distributed data store may maintain the historical transactional records, rate card data, and procedure-revenue profile matrix entries specific to its respective hospital. The per-hospital data isolation layer 1608 may enforce logical separation among the distributed data store A 1602, the distributed data store B 1604, and the distributed data store N 1606, such that transactional records and patient-level data belonging to one hospital may not be accessible to another hospital's processing pipeline. The per-hospital data isolation layer 1608 may facilitate multi-tenant deployment while preserving data confidentiality boundaries across hospital clients.

The shared model training layer 1610 may execute operations above the per-hospital data isolation layer 1608 and may aggregate de-identified or anonymized training signals from the distributed data stores 1602, 1604, 1606. The machine learning engines 112 and the reinforcement learning engine 208 may be updated in real-time. The shared model training layer 1610 may enable the association rule mining and the UCB-based weight update mechanisms to benefit from cross-hospital data volume without exposing hospital-specific records across the per-hospital data isolation layer 1608. The payer-specific profiles 1612 may store the payer-partitioned procedure-revenue profile matrix entries such that a given procedure code may have a different weight score for a first payer identifier than for a second payer identifier. The centralized rule updates 1614 may distribute updated validation rules from the NLP-based coding standards update module 420, propagating rule changes across all hospital deployments without requiring per-hospital manual intervention. The federated deployment module 1616 may coordinate the distribution of updated model weights, rule sets, and payer-specific profiles from the shared model training layer 1610, as well as centralized rule updates 1614, to each of the distributed data stores 1602, 1604, 1606.

The cloud infrastructure layer 1618 may provide the underlying platform services supporting the deployment architecture 1600. Data factory 1620 may correspond to the ETL pipeline that orchestrates data movement and transformation across hospital data sources.

In an embodiment, the per-hospital data isolation layer 1608 and the federated deployment module 1616 may be implemented using containerized microservices orchestrated by a container orchestration platform. Each hospital deployment may execute within an isolated container instance, and the container orchestration platform may manage scaling, load balancing, and service discovery across the distributed data stores 1602, 1604, 1606. The containerized architecture enables concurrent processing of claims from multiple hospital clients without cross-tenant data leakage and permits independent scaling of individual processing components, such as the machine learning engines 112 and the reinforcement learning engine 208, based on per-hospital claim volume.

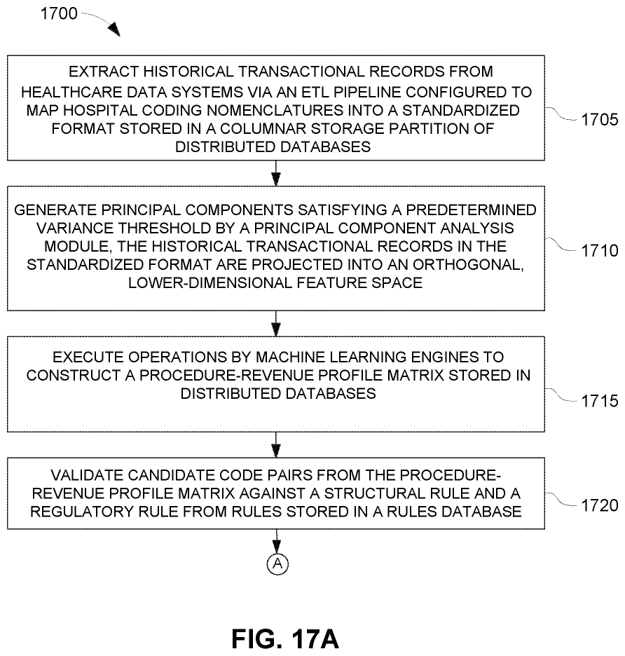

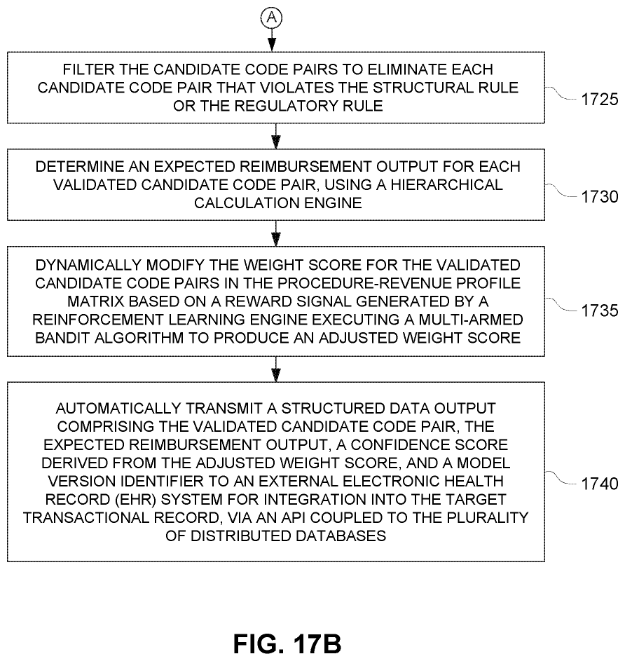

FIG. 17A and FIG. 17B illustrates a flow diagram showing a process 1700 implemented by the reinforcement-learning-driven profile matrix optimization system 110, according to an exemplary embodiment. FIG. 17A and FIG. 17B are described in conjunction with FIGS. 1-16.

At step 1705, multiple historical transactional records may be extracted from multiple healthcare data systems via the ETL pipeline, executed, and mapped to a standardized format stored in a columnar storage partition of distributed databases. Each historical transactional record in the standardized format may include, for example, the procedure code, revenue code, payer identifier, and transaction outcome value. The multiple healthcare data systems may use heterogeneous coding nomenclatures and data formats. The ETL pipeline at step 1705 may resolve the technical interoperability problem arising from heterogeneous healthcare data systems by normalizing the multiple hospital coding nomenclatures into a standardized format before storing historical transactional records in the columnar storage partition of the distributed databases. The columnar storage partition may include the claim history table 704, which may store the procedure code, revenue code, payer identifier, location, payment amount, and status for each historical transactional record. In an embodiment, the multiple historical transactional records may include records from multiple payers across multiple treatment locations, and the resulting combinatorial search space of procedure-revenue code pairings across payers and locations may be computationally intractable for manual or rule-based approaches.

At step 1710, multiple principal components satisfying a predetermined variance threshold may be generated by a principal component analysis (PCA) module. The multiple historical transactional records in the standardized format may be projected into an orthogonal, lower-dimensional feature space. The projection into the orthogonal, lower-dimensional feature space may reduce the dimensionality of the historical transactional records while retaining statistically significant variance. In an embodiment, the predetermined variance threshold may be, for example, 95%, and the projection may be applied to each partition of the plurality of historical transactional records defined by the payer identifier and the location identifier, thereby enabling dimensionality reduction on a per-payer, per-location basis. The principal components generated at step 1710 may capture the statistically significant correlations between procedure codes and revenue codes present in the historical transactional records while discarding noise and redundant dimensions.

At step 1715, multiple operations may be executed by machine learning engines to construct a procedure-revenue profile matrix stored in distributed databases. Construction of the procedure-revenue profile matrix at step 1715 may comprise identifying procedure codes, each paired with at least one revenue code, where the procedure codes have a mean transaction outcome value exceeding zero across records associated with non-zero transaction outcome values. Construction of the procedure-revenue profile matrix at step 1715 may further include mapping a usage metric and a weight score to a corresponding cell of the procedure-revenue profile matrix for each identified pairing of a procedure code and a revenue code. Each cell may be indexed for retrieval by a combination of the procedure code, the revenue code, and the payer identifier. The usage metric may correspond to the USAGE PERCENTAGE field, and the weight score may correspond to the WEIGHT SCORE field. In an embodiment, the machine learning engines at step 1715 may further execute association rule mining on the plurality of principal components generated at step 1710 to discover statistically significant correlations between procedure codes and revenue codes. In another embodiment, the machine learning engines at step 1715 may eliminate each identified pairing of a procedure code and a revenue code that is associated with fewer than the minimum sample count of records with non-zero transaction outcome values, where the minimum sample count may be auto-calculated by the machine learning engines. In another embodiment, the machine learning engines at step 1715 may execute integer programming to combinatorially optimize candidate code pairs derived from the procedure-revenue profile matrix, where the integer program maximizes the predicted outcome value across the candidate code pairs, subject to constraints derived from structural and regulatory rules.

At step 1720, candidate code pairs from the procedure-revenue profile matrix may be validated against structural rules and regulatory rules stored in a rules database. The structural rules may include location-specific constraints and dependency constraints between procedure codes and revenue codes. The regulatory rules may include rules derived from external coding standards maintained by regulatory bodies. In an embodiment, a location-constraint checker may apply the structural rule to verify whether a procedure code is permissible for a treatment location identified in a target transactional record. In an embodiment, a dependency-constraint checker may apply the structural rule to verify that revenue codes selected for a first procedure code on the target transactional record do not conflict with revenue codes selected for a second procedure code on the target transactional record. The validation at step 1720 may reference the validation rules table 710 of FIG. 7, where each rule may be stored with a RULE_ID, a RULE_TYPE, a PROC_CODE, a REV_CODE, a CONDITION, and a SOURCE.

At step 1725, as shown in FIG. 17B, the candidate code pairs may be filtered to eliminate each candidate code pair that violates the structural rule or the regulatory rule. Step 1725 may follow from the validation performed at step 1720. The filtering at step 1725 may produce a set of validated candidate code pairs that may have passed both structural and regulatory validation checks. In an embodiment, the filtering at step 1725 may further include classifying each validated candidate code pair into one of the categories, for example, auto-approved, auto-rejected, or flagged-for-manual-review. The classification may be based on a confidence score derived from the weight score and the usage metric stored in the procedure-revenue profile matrix. Candidate code pairs classified as flagged-for-manual-review may be presented to a revenue cycle analyst 216 through a user interface for acceptance, rejection, or modification.

At step 1730, a predicted outcome value may be determined for each validated candidate code pair, using a hierarchical calculation engine. Determining the predicted outcome value at step 1730 may include selecting a rate record from a rate data store based on a temporal attribute of a target transactional record and a priority hierarchy. The priority hierarchy may prioritize a plan-specific rate record over a location-specific rate record over a general rate record. Determining the predicted outcome value at step 1730 may further include applying a rate formula selected based on a combination of the payer identifier, a plan identifier, and a location identifier associated with the target transactional record. The rate formula may be selected from a per-case, per-diem, diagnosis-group-based, percentage-of-charges, per-unit, or fee-schedule formula. In an embodiment, the hierarchical calculation engine at step 1730 may further comprise a rate exception engine that evaluates exception rules spanning plans, service locations, and contracts stored in the rate data store.

At step 1735, the weight score for the validated candidate code pairs in the procedure-revenue profile matrix may be dynamically modified based on a reward signal generated by a reinforcement learning engine executing a multi-armed bandit algorithm to produce an adjusted weight score. The reinforcement learning engine at step 1735 may derive the reward signal from downstream outcome data by computing a composite function of an approval status and a transaction outcome associated with processed transactional records. The composite function may comprise a reimbursement component weighted by a first coefficient, an approval-indicator component weighted by a second coefficient, and a constraint-violation penalty component weighted by a third coefficient. The reinforcement learning engine at step 1735 may calculate an exploration-exploitation score for each validated candidate code pair, balancing an estimated cumulative reward against a degree of uncertainty derived from a ratio of the total count of selections across all validated candidate code pairs to a count of times the validated candidate code pair has been selected. The weight score may be updated by incrementally adjusting the weight score toward the reward signal in proportion to an inverse of the count of times the validated candidate code pair has been selected, using the weight update equation as described previously. In an embodiment, the multi-armed bandit algorithm at step 1735 may be a contextual bandit algorithm. In another embodiment, the reinforcement learning engine at step 1735 may receive relevance feedback from a domain analyst via a user interface coupled to the reinforcement learning engine, where the relevance feedback may comprise an acceptance, a rejection, or a modification of validated candidate code pairs, and the relevance feedback may be included as a ground truth label into the reward signal to update the weight score in the procedure-revenue profile matrix.

At step 1740, a structured data output may be automatically transmitted. For example, the structured data output may include the validated candidate code pair, the predicted outcome value, a confidence score derived from the adjusted weight score, and a model version identifier, all integrated into the target transactional record via an API coupled to the multiple distributed databases. The structured data output at step 1740 may be transmitted for integration into the target transactional record, such that the external data system may receive the validated candidate code pair and associated metadata for downstream processing. In an embodiment, the structured data output at step 1740 may be stored in a tamper-resistant audit data store.

In another embodiment, the process 1700 may further comprise executing a sandbox testing simulation by applying the validated candidate code pairs to a subset of the historical transactional records, and comparing the predicted outcome value for each validated candidate code pair against the actual transaction outcome value recorded in a corresponding historical transactional record in the ranked subset. In response to the detection or determination of a deviation between the predicted outcome value and the actual transaction outcome value exceeding a predefined acceptance threshold for more than a predefined proportion of the ranked subset, automatically retrieve a prior version of the procedure-revenue profile matrix via a version control mechanism and replace the procedure-revenue profile matrix with the prior version.

In an embodiment, the data processing system (e.g., reinforcement-learning-driven profile matrix optimization system 110) may execute operations to extract multiple historical transactional records from multiple healthcare data systems via the ETL pipeline. The ETL pipeline executes operations to map the multiple hospital coding nomenclatures into a standardized format stored in columnar storage partitions of distributed databases. Each transactional record in the standardized format includes a procedure code, a revenue code, a payer identifier, and a transaction outcome value. The ETL pipeline uses Azure Data Factory to connect to hospital information systems. The pipeline may receive claims data, for example, in HL7 v2. x format for real-time ADT feeds and X12 837 format for batch claim submissions. During transformation, the data processing system may normalize varying hospital coding nomenclatures, for example, mapping the internal revenue code labels and the procedure code identifiers into a unified schema including standardized fields for procedure code, revenue code, payer identifier, location, patient class, admission date, service date, plan identifier, DRG code, and charge amount. The standardized records are stored using columnar storage partitions, which may accelerate analytical aggregation queries for downstream machine learning engines.

The PCA module in the data processing system generates multiple principal components that satisfy a predetermined variance threshold. The PCA module projects the multiple historical transactional records into a standardized, orthogonal, lower-dimensional feature space. Such a projection reduces the high-dimensional input feature set while retaining the statistically significant variance in the data. The PCA module applies per payer and per location partition, preserving payer-specific billing patterns during dimensionality reduction. The PCA module may retain, for example, approximately 10 to 12 principal components that satisfy a variance threshold of at least 95%. In various embodiments, the PCA module may retain at least 5, at least 8, at least 10, or at least 12 principal components.

The data processing system executes operations by implementing machine learning engines to construct the PRP matrix and store it in distributed databases. The construction proceeds by identifying procedure codes, each paired with a revenue code, for which the mean transaction outcome value exceeds zero across records with non-zero transaction outcome values. This filtering includes only combinations with a history of positive reimbursement in the matrix. The data processing system also eliminates each identified pairing with fewer than the minimum sample count of records with non-zero transaction outcome values. The minimum sample count is auto-calculated by the machine learning engines. The pairings associated with fewer than three sample records are pruned to avoid statistical noise from sparse data. For each identified pairing of a procedure code and a revenue code, the system maps a usage metric and a weight score to a corresponding cell of the procedure-revenue profile matrix. Each cell is indexed for retrieval by a combination of the procedure code, the revenue code, and the payer identifier.

For example, for procedure code 99213 (office visit, established patient) paired with payer Blue Cross at location 001, the PRP matrix cell stores a usage percentage of 78.4%, an approval rate of 92.1%, an average reimbursement of $142.50, and a weight of 1.247. The PRP matrix schema may include columns for profile identifier, procedure code, revenue code, payer identifier, location identifier, patient class, usage percentage, approval rate, average reimbursement, weight, effective start date, effective end date, last updated timestamp, and model version, with a unique constraint on the combination of procedure code, revenue code, payer identifier, and location identifier. The technical advantage is that the indexed PRP matrix enables constant-time lookups at runtime for a given claim's procedure code, payer identifier, and location.

The machine learning engines further execute integer programming to optimize combinatorially the candidate code pairs derived from the procedure-revenue profile matrix. The integer programming formulation maximizes the predicted outcome value across candidate code pairs, subject to constraints derived from structural rules and regulatory rules. For a claim with ‘N’ procedure codes, each having ‘M’ possible revenue codes, the theoretical search space is M^{N} combinations. Integer programming reduces this combinatorial explosion by pruning infeasible branches using constraint satisfaction, yielding computationally tractable solutions. In the example shown in FIG. 9, a sample claim with three procedures, P1 (CPT 43239) with two revenue code options, P2 (CPT 43249) with two options, and P3 (CPT 76000) with three options, generates 12 valid combinations (2×2×3). The integer programming engine evaluates each combination through the iPRC and selects the optimal combination. The technical advantage is that integer programming guarantees a globally optimal solution in the feasible region, whereas heuristic methods may converge to local optima.

The data processing system validates candidate code pairs from the procedure-revenue profile matrix against structural and regulatory rules stored in a rules database. Based on validation, the system filters out candidate code pairs that violate the structural or regulatory rules. The structural rules are enforced by a domain-specific rules engine comprising a procedure-location validity checker and an inter-procedure dependency constraint checker. The procedure-location validity checker applies the structural rule to verify that a given procedure code is permissible for the treatment location identified in the target transactional record. For example, a GI procedure assigned to revenue code 0360 (e.g., operating room) may be valid, but the same procedure assigned to a general treatment room may violate a location constraint. The dependency constraint checker verifies that the revenue codes selected for a first procedure code on the target transactional record do not conflict with the revenue codes selected for a second procedure code on the same record. The technical advantage of this two-layer validation, structural and regulatory, is that the system determines both domain-specific coding errors and regulatory non-compliance before the candidate code pairs reach the iPRC for reimbursement calculation.

The validation further classifies each validated candidate code pair as auto-approved, auto-rejected, or flagged-for-manual-review, based on a confidence score derived from the weight score and the usage metric stored in the procedure-revenue profile matrix. A candidate code pair with a confidence score above a high threshold is auto-approved. A pair with a confidence score below a low threshold is auto-rejected. A pair falling between the two thresholds is routed to a manual review queue and flagging engine, where a revenue cycle analyst reviews it through the user interface. The technical advantage is that the three-tier classification accelerates claims processing for high-confidence combinations while preserving human oversight for ambiguous cases.

The data processing system determines a predicted outcome value for each validated candidate code pair using a hierarchical calculation engine. The determination proceeds by selecting a rate record from a rate data store based on a temporal attribute of the target transactional record and a priority hierarchy, and then applying a rate formula selected based on a combination of the payer identifier, plan identifier, and location identifier associated with the target transactional record. The priority hierarchy prioritizes a plan-specific rate record over a location-specific rate record, and both over a general rate record. The rate formula is selected from a per-case formula, a per-diem formula, a diagnosis-group-based formula, a percentage-of-charges formula, a per-unit formula, and a fee-schedule formula.

For example, a per-case formula computes Payment=BaseRate*DRG_Weight*LocationAdjustment. The per-diem formula computes Payment=DailyRate*LengthOfStay, capped at the contractual maximum. For example, with a daily rate of $1,200 per day, a length of stay of 5 days, and a contractual cap of $5,500, the computed payment is min($6,000, $5,500)=$5,500. The percentage-of-charges formula computes Payment=TotalCharges*ContractedPercentage. The fee-schedule formula computes Payment=FeeScheduleRate (ProcedureCode, Location, EffectiveDate). The hierarchical calculation engine further comprises a rate exception engine that evaluates exception rules spanning plans, service locations, and contracts stored in the rate data store.

The rate exception engine processes, for example, more than 772,546 health plan rules across 71 plans, 12 locations, and 39 contracts. The rate card resolution engine executes operations for contract matching by effective date and plan, resolves multi-plan conflicts, and feeds the selected rate into the payment methodology selector. The hierarchical calculation engine applies priority ranking and payment aggregation to produce the expected reimbursement per combination. The technical advantage is that hierarchical rate resolution with a priority cascade manages the complexity of overlapping contracts and multi-plan conflicts without requiring manual adjudication.

The data processing system dynamically adjusts the weight score for validated candidate code pairs in the procedure-revenue profile matrix based on a reward signal generated by a reinforcement learning engine that executes a multi-armed bandit algorithm to produce the adjusted weight score. The reinforcement learning engine derives the reward signal from downstream outcome data by computing a composite function of the approval status and the transaction outcome for processed transactional records. The multi-armed bandit algorithm is a contextual bandit algorithm. The state space is defined by the contextual features S={procedure code, payer identifier, location, patient class, service date range}. The action space is the set of valid revenue codes for a given procedure code as determined by the PRP matrix. The reinforcement learning engine calculates an exploration-exploitation score for each validated candidate code pair. The exploration-exploitation score balances an estimated cumulative reward for the validated candidate code pair against a degree of uncertainty derived from the ratio of the total count of selections across all validated candidate code pairs to the count of times the validated candidate code pair has been selected.

The reinforcement learning engine updates the weight score for the validated candidate code pair by incrementally adjusting it toward the reward signal, in proportion to the inverse of the number of times the validated candidate code pair has been selected. The initial weights are derived from two years of historical approval rates per procedure-revenue-payer combination. The technical advantage is that the contextual bandit continuously adapts the PRP matrix to reflect evolving payer behavior and reimbursement patterns without requiring full model retraining.

The reward signal is a composite function comprising a reimbursement component weighted by a first coefficient α, an approval-indicator component weighted by a second coefficient β, and a constraint-violation penalty component weighted by a third coefficient γ. In an embodiment, using equation (1) with α=0.6, β=0.3, and γ=0.1, the coefficients are configurable and initialized from the hierarchical contract rules. The reimbursement component captures financial optimization. The approval-indicator component, which is binary (e.g., 1 for approved, 0 for denied), ensures that the data processing system does not optimize purely for revenue at the cost of claim denials. The constraint-violation penalty discourages recommendations that, while financially attractive, approach regulatory boundaries. The technical advantage is that the composite reward function prevents the reinforcement learning engine from converging on high-reimbursement combinations that have high denial rates.

The data processing system automatically transmits a structured data output comprising the validated candidate code pair, the predicted outcome value, a confidence score derived from the adjusted weight score, and a model version identifier to a data system for integration into the target transactional record, via an API coupled to the distributed databases. The API exposes a RESTful endpoint that accepts claim details as input and returns the ranked code recommendations. The structured data output is formatted as a JSON payload including the recommended procedure-revenue code pair, the computed expected reimbursement, the confidence score, and the model version string. The external data system ingests this payload and updates the target transactional record accordingly. The technical advantage is that automated transmission via a structured API eliminates the need to re-enter recommendations and ensures traceability via the model version identifier.

The procedure-revenue profile matrix is stored in the columnar storage partitions as a partitioned data structure comprising fields associated with the usage metric, the weight score, an exploration-exploitation score computed by the reinforcement learning engine for each validated candidate code pair, an effective date, and a status indicator. The procedure-revenue profile matrix is partitioned by the payer identifier such that a given procedure code has a different weight score for a first payer identifier than for a second payer identifier.

The data processing system executes a sandbox testing simulation on a selected subset of the plurality of historical transactional records. The data processing system applies the validated candidate code pairs to the selected subset. The data processing system compares the expected reimbursement amount for each validated candidate code pair with the actual reimbursement amount from the corresponding historical transaction record in the selected subset. In response to detection or determination of a deviation between the predicted outcome value and the actual transaction outcome value exceeding a predefined acceptance threshold for more than a predefined proportion of historical transactional records in the selected subset, the data processing system retrieves a prior version of the procedure-revenue profile matrix via a version control mechanism and replaces the procedure-revenue profile matrix with the prior version to maintain system stability.

The sandbox is a parallel instance of the production system operating on the most recent six months of historical claims, ranked by payer, procedure type, and location. The acceptance threshold is set such that predicted reimbursement must fall in ±5% of actual reimbursement for more than 90% of test claims. The version control mechanism maintains timestamped snapshots of the PRP matrix. If the new model version underperforms on any key metric by more than 2% in an A/B comparison against the current production version, automatic rollback to the prior version is triggered. The technical advantage is that sandbox testing with automated rollback prevents untested model changes from degrading production accuracy.

The data processing system stores, in a tamper-resistant audit data store, an audit record for each validated candidate code pair. The audit record comprises an original code assignment of the target transactional record, a recommended code pair corresponding to the validated candidate code pair, a confidence score, a model version identifier, and a timestamp. Tamper resistance is achieved through append-only write policies and cryptographic hash chaining, ensuring that no audit entry can be retroactively altered. The technical advantage is that the audit trail satisfies HIPAA compliance requirements for healthcare data systems and enables full reproducibility of the reasoning behind any historical recommendation.

In an embodiment, the data processing system monitors, via the API, an external data source for an update to external rules. Monitoring the external data source comprises executing an NLP pipeline that includes named entity recognition and text classification to parse unstructured text from updated coding manuals, extract modified rule parameters, and dynamically map the extracted parameters to the rules database without disrupting ongoing claims processing.

The NLP-based coding standards update module connects to external coding standards APIs from CMS, AMA, and state agencies, as shown in the external coding standards API. The change detection module identifies modified sections in coding manual updates by comparing incoming documents with prior versions using text-similarity scoring. The NLP entity extraction module applies named entity recognition to extract procedure code identifiers, revenue code references, effective dates, and rule modifications from the semi-structured documents. Text classification categorizes each extracted change as a new rule, a modification to an existing rule, or a deprecation. The extracted parameters are mapped to existing rules in the rules database. Modified rules proceed through sandbox validation, expert review, and approval before production deployment. The technical advantage is that automated monitoring with NLP-based extraction eliminates the lag between the publication of coding standard updates and their integration into the system's validation rules, thereby reducing the risk that claims processed under outdated rules are denied.

The data processing system receives, via a user interface coupled to the reinforcement learning engine, relevance feedback from a domain analyst. Relevance feedback comprises acceptance, rejection, or modification of a validated candidate code pair. The data processing system uses relevance feedback as a ground-truth label in the reward signal to update the weight scores in the procedure-revenue profile matrix. The user interface is a PowerApps-based action tracker that presents each flagged-for-manual-review candidate code pair with the confidence score, the original code assignment, and the predicted outcome value. The analyst's decision, including accept, reject, or modify, is recorded as a ground-truth label. This label is fed back to the reinforcement learning engine's reward calculation module as an additional input to the reward function. The technical advantage is that analyst feedback injects domain expertise into the reinforcement learning loop, correcting for cases where statistical optimization alone may not capture clinical or regulatory nuance.

The projection into the orthogonal, lower-dimensional feature space is applied for each partition of the multiple historical transactional records defined by the payer identifier and the location identifier. The predetermined variance threshold, for example, 95%. Per-payer, per-location PCA partitioning ensures that the principal components capture the billing variance structure specific to each payer-location combination rather than averaging across heterogeneous payer populations.

To illustrate the end-to-end operation of the data processing system, let us consider the following example. A mid-size hospital network processes claims across 40 payers, 15 locations, 5,000 procedure codes, and 800 revenue codes. The ETL pipeline extracts 18 months of historical claims data from three healthcare data systems, normalizing their coding nomenclatures into the standardized format. The PCA module applies per payer-location partition, retaining twelve principal components at a 95% variance threshold. The ML engines construct the PRP matrix by identifying all procedure-revenue code pairings with a mean reimbursement exceeding zero and three supporting records. For procedure code 43239 (upper GI endoscopy with biopsy) with payer Aetna at location 001, the PRP matrix stores two revenue code options, for example, revenue code 0360 (operating room) at 70% usage with weight 1.35, and revenue code 0750 (GI lab) at 30% usage with weight 0.92. The validation module applies the structural rule that procedure 43239 is permissible at treatment location 001 (e.g., an endoscopy suite) and verifies that there is no inter-procedure dependency conflict with concurrent procedure 43249 on the same claim. The regulatory compliance engine confirms compliance with CMS and AMA coding guidelines. The confidence score for the 0360 pairing is 0.87, exceeding the high threshold, so it is auto-approved. The iPRC selects the plan-specific rate record for the Aetna PPO-Gold plan effective on the service date, applies the fee-schedule formula, and computes an expected reimbursement of $4,200 for the 0360 pairing versus $3,100 for the 0750 pairing. The integer programming optimizer evaluates all 12 combinations for the three-procedure claim and selects the combination that maximizes total expected reimbursement at $11,200, compared to the original coding at $8,500, an improvement of $2,700 (31.8%). The reinforcement learning engine, implementing a contextual bandit with UCB, computes the exploration-exploitation score for each pairing and updates the weight using Q_new (0360)=1.35+ (1/47)×(0.91−1.35)=1.341, where 47 is the number of prior selections and 0.91 is the composite reward signal (0.6×1.0+0.3×1−0.1×0=0.9, normalized). The structured data output—including the recommended code pair (43239, 0360), expected reimbursement $4,200, confidence 0.87, and model version “v2.4.1”, is transmitted via the API to the hospital's external data system. The audit trail records the original assignment (43239, 0750), the recommendation (43239, 0360), confidence 0.87, model version v2.4.1, and timestamp. If a CMS update modifies the reimbursement rules for endoscopy procedures, the NLP pipeline's NER module extracts the updated rule parameters, maps them to the affected entries in the rules database, validates the changes in the sandbox against the most recent six months of claims, and deploys the updated rules to production after expert approval. Sandbox testing confirms that the updated model produces expected reimbursements in ±5% of actual for 93% of test claims, exceeding the 90% acceptance threshold. Should a subsequent model update cause the deviation to exceed the threshold, the version control mechanism rolls back to the prior PRP matrix snapshot to maintain system stability.

The computational operations described herein including projection of multi-dimensional transactional data into an orthogonal lower-dimensional feature space via principal component analysis, construction and continuous refinement of a sparse matrix indexed across three or more dimensions with cell-level weight updates managed by a bandit algorithm, and hierarchical rate resolution across temporally overlapping contract windows may include spanning hundreds of thousands of rules cannot practically be performed in the human mind. The data processing system executes operations on datasets including millions of historical transactional records across dozens of payers and locations simultaneously, producing validated candidate code pairs with associated expected transaction outcome values, confidence scores, and audit metadata within processing windows that are orders of magnitude shorter than any manual alternative. The data processing system architecture described herein may implement integrated computational mechanisms to solve a problem whose scale exceeds human cognitive capacity. In various embodiments, the data processing system may process at least 100,000, at least 500,000, at least 1,000,000, at least 5,000,000, or at least 10,000,000 historical transactional records. In various embodiments, the data processing system may process at least 1,000, at least 5,000, at least 10,000, at least 50,000, or at least 100,000 claims in no more than 1 minute, in no more than 5 minutes, or in no more than 10 minutes. In various embodiments, the combinatorial claims optimizer engine may evaluate at least 100,000, at least 500,000, at least 1,000,000, or at least 10,000,000 candidate code combinations in no more than 1 second, in no more than 5 seconds, or in no more than 10 seconds. In various embodiments, the procedure-revenue profile matrix lookup may return results for a given procedure-payer-location combination within no more than 10 milliseconds, no more than 50 milliseconds, or no more than 100 milliseconds. In various embodiments, the code combination validation module may validate a candidate code pair against at least 1,000, at least 10,000, or at least 100,000 rules within 100 milliseconds, 500 milliseconds, or 1 second. In various embodiments, the rate exception engine may evaluate at least 100,000, at least 500,000, or at least 750,000 health plan rules in no more than 1 second; in no more than 5 seconds, or in no more than 10 seconds. In various embodiments, the reinforcement learning engine may update weight scores for at least 1,000, at least 10,000, or at least 100,000 candidate code pairs within 1 second, 5 seconds, or 10 seconds.

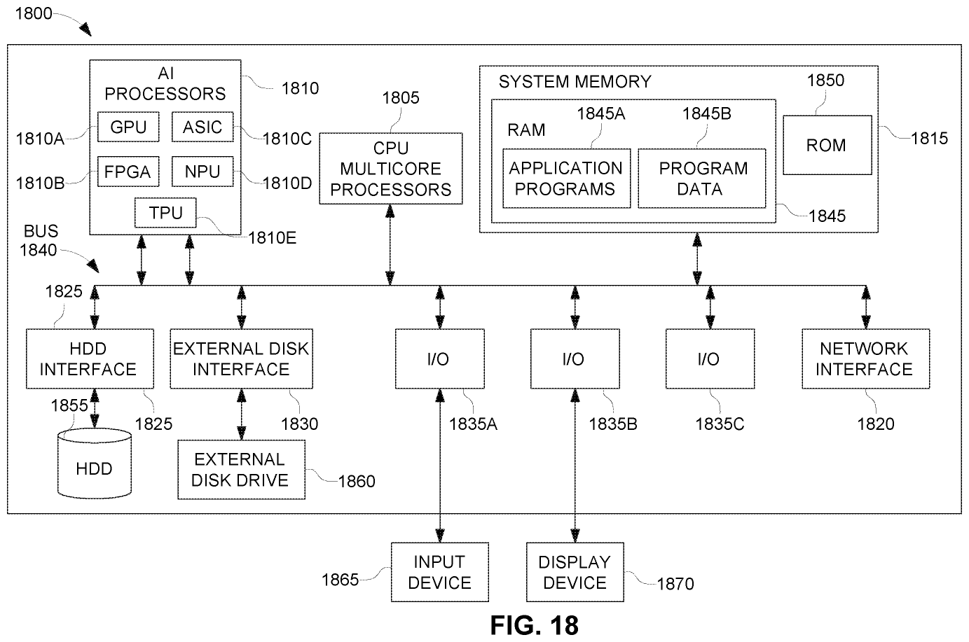

FIG. 18 illustrates an exemplary hardware configuration of a computer 1800 used to implement the system and method described herein, according to exemplary embodiments. The computer 1800 shown in FIG. 18 includes CPU 1805, GPU 1810, system memory 1815, network interface 1820, hard disk drive (HDD) interface 1825, external disk drive interface 1830, and input/output (I/O) interfaces 1835A, 1835B, 1835C. These elements of the computer are coupled to each other via the system bus 1840. The CPU 1805 may perform arithmetic, logic, and/or control operations by accessing system memory 1815. The CPU 1805 may implement the processors of the exemplary devices and/or system described above. The GPU 1810 may perform graphics or AI processing operations. In case computer 1800 is used to implement an exemplary central processing device, GPU 1810 may be the GPU 1810 of the exemplary central processing device as described above. The GPU 1810 may further include or be supplemented by additional AI processors such as an FPGA 1810A, an ASIC 1810B, an NPU 1810C, a TPU 1810D, and multicore processors 1810E. The computer 1800 does not necessarily include the GPU 1810, for example, if computer 1800 is used to implement a device other than a central processing device. The system memory 1815 may store information and/or instructions for use in combination with the CPU 1805. The information and/or instructions may be used to implement the execution of the engines described in the system and method in the subject specification. The system memory 1815 may include volatile and non-volatile memory, such as random-access memory (RAM) 1845 and read-only memory (ROM) 1850. A basic input/output system (BIOS), including the basic routines that help to transfer information between elements in the computer 1800, such as during start-up, may be stored in ROM 1850. The system bus 1840 may be any of several types of bus structures, including a memory bus or memory controller, a peripheral bus, and a local bus using any of a variety of bus architectures. The computer may include a network interface 1820 for communicating with other computers and/or devices via a network.

Further, the computer may include a hard disk drive (HDD) 1855 for reading from and writing to a hard disk (not shown), and an external disk drive 1860 for reading from or writing to a removable disk (not shown). The removable disk may be a magnetic disk for a magnetic disk drive or an optical disk, such as a CD-ROM for an optical disk drive. The HDD 1855 and external disk drive 1860 are connected to the system bus 1840 via the HDD interface 1825 and the external disk drive interface 1830, respectively. The drives and their associated non-transitory computer-readable media provide non-volatile storage of computer-readable instructions, data structures, program modules, and other data for the general-purpose computer. The relevant data may be organized in a database, for example, a relational or object database.

Although the exemplary environment described herein employs a hard disk (not shown) and an external disk (not shown), it should be appreciated by those skilled in the art that other types of computer readable media which can store data that is accessible by a computer, such as magnetic cassettes, flash memory cards, digital video disks, random access memories, read only memories, and the like, may also be used in the exemplary operating environment.

Several program modules may be stored on the hard disk, external disk, ROM 1850, or RAM 1845, including an operating system (not shown), one or more application programs 1845A, other program modules (not shown), and program data 1845B. The application programs may include at least a part of the functionality as described above.

The computer 1800 may be connected to input device 1865, such as a mouse and/or keyboard, and display device 1870, such as a liquid crystal display, via corresponding I/O interfaces 1835A to 1835C and the system bus 1840. In addition to an implementation using a computer 1800 as shown in FIG. 18, a part or all of the functionality of the exemplary embodiments described herein may be implemented as one or more hardware circuits. Examples of such hardware circuits may include, but are not limited to, Large Scale Integration (LSI), Reduced Instruction Set Circuits (RISC), Application Specific Integrated Circuit (ASIC), and Field Programmable Gate Array (FPGA).

One or more embodiments are now described with reference to the drawings, wherein like reference numerals are used to refer to like elements throughout. In the following description, for purposes of explanation, numerous specific details are set forth to provide a thorough understanding of the various embodiments. It is evident, however, that the various embodiments can be practiced without these specific details (and without applying to any networked environment or standard).

As used in this application, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both the application running on a server and the server can be components.

The above descriptions and illustrations of embodiments, including what is described in the Abstract, are not intended to be exhaustive or to limit the one or more embodiments to the precise forms disclosed. While specific embodiments of, and examples for, the one or more embodiments are described herein for illustrative purposes, various equivalent modifications are possible in the scope, as those skilled in the relevant art will recognize. These modifications can be made based on the detailed description above. Rather, the scope is to be determined by the following claims, which are to be interpreted in accordance with established doctrines of claim construction.

What is claimed is:

1. A data processing system, comprising:
a processor; and
a non-transitory computer-readable memory coupled to the processor and storing instructions that, when executed by the processor, cause the data processing system to:
extract a plurality of historical transactional records from a plurality of healthcare data systems via an Extract, Transform, Load (ETL) pipeline configured to map a plurality of hospital coding nomenclatures into a standardized format stored in one or more columnar storage partitions of a plurality of distributed databases, wherein the standardized format includes at least a procedure code, a revenue code, a payer identifier, and a transaction outcome value;
generate a plurality of principal components satisfying a predetermined variance threshold by a principal component analysis module, wherein the plurality of historical transactional records in the standardized format are projected into an orthogonal, lower-dimensional feature space;
execute one or more operations by one or more machine learning engines to construct a procedure-revenue profile matrix stored in one or more distributed databases by:
identifying one or more procedure codes, each paired with at least one revenue code, the one or more procedure codes having a mean transaction outcome value exceeding zero across records associated with non-zero transaction outcome values; and
for each identified pairing of a procedure code and a revenue code, mapping a usage metric and a weight score to a corresponding cell of the procedure-revenue profile matrix, wherein each cell is indexed for retrieval by a combination of the procedure code, the revenue code, and the payer identifier;
validate one or more candidate code pairs from the procedure-revenue profile matrix against at least one structural rule and at least one regulatory rule from a plurality of rules stored in a rules database;
based on the validation, filter the one or more candidate code pairs to eliminate each candidate code pair that violates the at least one structural rule or the at least one regulatory rule;
determine a predicted outcome value for each validated candidate code pair, using a hierarchical calculation engine, by:
selecting a rate record from a rate data store based on a temporal attribute of a target transactional record and a priority hierarchy; and
applying a rate formula selected based on a combination of the payer identifier, a plan identifier, and a location identifier associated with the target transactional record;
dynamically adjust the weight score for one or more validated candidate code pairs in the procedure-revenue profile matrix based on a reward signal generated by a reinforcement learning engine executing a multi-armed bandit algorithm to produce an adjusted weight score, the reinforcement learning engine deriving the reward signal from downstream outcome data by computing a composite function of an approval status and a transaction outcome associated with one or more processed transactional records, wherein the downstream outcome data comprises an approval status and a transaction outcome associated with one or more processed transactional records; and
automatically transmit a structured data output comprising the validated candidate code pair, the predicted outcome value, a confidence score derived from the adjusted weight score, and a model version identifier to an external data (e.g., healthcare-related data) system for integration into the target transactional record, via an API coupled to the plurality of distributed databases.

2. The data processing system of claim 1, wherein the projection into the orthogonal, lower-dimensional feature space is applied for each partition of the plurality of historical transactional records defined by the payer identifier and the location identifier, and wherein the predetermined variance threshold is at least 95%.

3. The data processing system of claim 1, wherein the identifying further comprises eliminating each identified pairing of one or more procedure codes and one or more revenue codes that is associated with fewer than a minimum sample count of records associated with non-zero transaction outcome values, and wherein the minimum sample count is auto-calculated by the one or more machine learning engines.

4. The data processing system of claim 1, wherein the one or more machine learning engines further execute integer programming for combinatorial optimization of the one or more candidate code pairs derived from the procedure-revenue profile matrix.

5. The data processing system of claim 1, wherein the target transactional record comprises
a first procedure code and a second procedure code, and wherein the validating comprises:
applying, by a location-constraint checker, the at least one structural rule to verify that the first procedure code is permissible for a treatment location identified in the target transactional record; and
applying, by a dependency-constraint checker, the at least one structural rule to verify that the one or more revenue codes selected for the first procedure code on the target transactional record do not conflict with the one or more revenue codes selected for the second procedure code on the target transactional record.

6. The data processing system of claim 1, wherein the validating further comprises classifying each validated candidate code pair into one of auto-approved, auto-rejected, or flagged-for-manual-review, based on a confidence score derived from the weight score and the usage metric stored in the procedure-revenue profile matrix.

7. The data processing system of claim 1, wherein the priority hierarchy prioritizes a plan-specific rate record over a location-specific rate record over a general rate record, and wherein the rate formula is selected from at least two of: a per-case formula, a per-diem formula, a diagnosis-group-based formula, a percentage-of-charges formula, a per-unit formula, and a fee-schedule formula.

8. The data processing system of claim 1, wherein the hierarchical calculation engine further comprises a rate exception engine that evaluates a plurality of exception rules spanning a plurality of plans, a plurality of service locations, and a plurality of contracts stored in the rate data store.

9. The data processing system of claim 1, wherein the multi-armed bandit algorithm is a contextual bandit algorithm, and wherein the reinforcement learning engine calculates an exploration-exploitation score for each validated candidate code pair, the exploration-exploitation score balancing an estimated cumulative reward for the validated candidate code pair against a degree of uncertainty derived from a ratio of a total count of selections across all validated candidate code pairs to a count of times the validated candidate code pair has been selected, and wherein the reinforcement learning engine updates the weight score for the validated candidate code pair by incrementally adjusting the weight score toward the reward signal in proportion to an inverse of the count of times the validated candidate code pair has been selected.

10. The data processing system of claim 1, wherein the reward signal is a composite function comprising a reimbursement component weighted by a first coefficient, an approval-indicator component weighted by a second coefficient, and a constraint-violation penalty component weighted by a third coefficient.

11. The data processing system of claim 1, wherein the instructions further cause the data processing system to:
execute a sandbox testing simulation on a selected subset of the plurality of historical transactional records;
apply the validated candidate code pairs to the selected subset;
compare the predicted outcome value determined for each validated candidate code pair against an actual transaction outcome value associated with a corresponding historical transactional record in the selected subset; and
in response to a deviation between the predicted outcome value and the actual transaction outcome value exceeding a predefined acceptance threshold for more than a predefined proportion of historical transactional records in the selected subset, retrieve a prior version of the procedure-revenue profile matrix via a version control mechanism and replace the procedure-revenue profile matrix with the prior version to maintain system stability.

12. The data processing system of claim 1, wherein the instructions further cause the data processing system to store, in a tamper-resistant audit data store, an audit record for each validated candidate code pair, the audit record comprising at least an original code assignment of the target transactional record, a recommended code pair corresponding to the validated candidate code pair, a confidence score, a model version identifier, and a timestamp.

13. The data processing system of claim 1, wherein the instructions further cause the data processing system to monitor, via the API, at least one external data source for an update to one or more external rules, wherein monitoring the at least one external data source comprises:
executing a natural language processing (NLP) pipeline comprising named entity recognition and text classification to parse an unstructured text of updated coding manuals;
extracting one or more modified rule parameters, and dynamically mapping the extracted parameters to the rules database without disrupting ongoing claims processing.

14. The data processing system of claim 1, wherein the procedure-revenue profile matrix is stored in the one or more columnar storage partitions as a partitioned data structure comprising fields associated with the usage metric, the weight score, an exploration-exploitation score computed by the reinforcement learning engine for each validated candidate code pair, an effective date, and a status indicator, and wherein the procedure-revenue profile matrix is partitioned by the payer identifier such that a given procedure code has a different weight score for a first payer identifier than for a second payer identifier.

15. The data processing system of claim 1, wherein the instructions further cause the data processing system to:
receive, via a user interface coupled to the reinforcement learning engine, relevance feedback from a domain analyst, wherein the relevance feedback comprises an acceptance, a rejection, or a modification of at least one of the validated candidate code pairs; and
incorporate the relevance feedback as a ground truth label into the reward signal to update the weight score in the procedure-revenue profile matrix.

16. A computer-implemented method executed by a processor, the method comprising:
extracting a plurality of historical transactional records from a plurality of healthcare data systems via an Extract, Transform, Load (ETL) pipeline configured to map a plurality of hospital coding nomenclatures into a standardized format stored in one or more columnar storage partitions of a plurality of distributed databases, wherein each historical transactional record in the standardized format includes at least a procedure code, a revenue code, a payer identifier, and a transaction outcome value;
generating a plurality of principal components satisfying a predetermined variance threshold by a principal component analysis module, wherein the plurality of historical transactional records in the standardized format are projected into an orthogonal, lower-dimensional feature space;
executing one or more operations by one or more machine learning engines to construct a procedure-revenue profile matrix stored in one or more distributed databases, by:
identifying one or more procedure codes, each paired with at least one revenue code, the one or more procedure codes having a mean transaction outcome value exceeding zero across records associated with non-zero transaction outcome values; and
for each identified pairing of a procedure code and a revenue code, mapping a usage metric and a weight score to a corresponding cell of the procedure-revenue profile matrix, wherein each cell is indexed for retrieval by a combination of the procedure code, the revenue code, and the payer identifier;
validating one or more candidate code pairs from the procedure-revenue profile matrix against at least one structural rule and at least one regulatory rule from a plurality of rules stored in a rules database;
based on the validation, filtering the one or more candidate code pairs to eliminate each candidate code pair that violates the at least one structural rule or the at least one regulatory rule;
determining a predicted outcome value for each validated candidate code pair, using a hierarchical calculation engine, by:
selecting a rate record from a rate data store based on a temporal attribute of a target transactional record and a priority hierarchy; and
applying a rate formula selected based on a combination of the payer identifier, a plan identifier, and a location identifier associated with the target transactional record;
dynamically adjusting the weight score for one or more validated candidate code pairs in the procedure-revenue profile matrix based on a reward signal generated by a reinforcement learning engine executing a multi-armed bandit algorithm to produce an adjusted weight score, the reinforcement learning engine deriving the reward signal from downstream outcome data by computing a composite function of an approval status and a transaction outcome associated with one or more processed transactional records, wherein the downstream outcome data comprises an approval status and a transaction outcome associated with one or more processed transactional records; and
automatically transmitting a structured data output comprising the validated candidate code pair, the predicted outcome value, a confidence score derived from the adjusted weight score, and a model version identifier to an external data (e.g., healthcare-related data) system for integration into the target transactional record, via an API coupled to the plurality of distributed databases.

17. The computer-implemented method of claim 16, wherein the projection into the orthogonal, lower-dimensional feature space is applied for each partition of the plurality of historical transactional records defined by the payer identifier and the location identifier, and wherein the predetermined variance threshold is at least 95%.

18. The computer-implemented method of claim 16, further comprising: executing, by the one or more machine learning engines, integer programming for combinatorial optimization of the one or more candidate code pairs derived from the procedure-revenue profile matrix, wherein the integer programming maximizes the predicted outcome value across the one or more candidate code pairs subject to constraints derived from the at least one structural rule and the at least one regulatory rule.

19. The computer-implemented method of claim 16, further comprising:
executing a sandbox testing simulation by applying the validated candidate code pairs to a ranked subset of the plurality of historical transactional records;
comparing the predicted outcome value for each validated candidate code pair against an actual transaction outcome value recorded in a corresponding historical transactional record of the ranked subset; and
in response to a deviation between the predicted outcome value and the actual transaction outcome value exceeding a predefined acceptance threshold for more than a predefined proportion of the ranked subset, automatically retrieving a prior version of the procedure-revenue profile matrix via a version control mechanism and replacing the procedure-revenue profile matrix with the prior version.

20. A non-transitory computer-readable medium storing instructions that, when executed by a processor, cause the processor to perform the method steps of claim 16.
